(19) United States
(12) Patent Application Publication
(10) Pub. No.: US 2003/0190612 A1
(43) Pub. Date: Oct. 9, 2003

(54) Detecting method and detection substrate for use therein

(76) Inventors: Nobuko Yamamoto, Kanagawa (JP); Tadashi Okamoto, Kanagawa (JP); Satoshi Shimizu, Kanagawa (JP); Tomohiro Suzuki, Kanagawa (JP)

Correspondence Address: FITZPATRICK CELLA HARPER & SCINTO, 30 ROCKEFELLER PLAZA, NEW YORK, NY, 10112, US

(21) Appl. No.: 09/942,662

(22) Filed: Aug. 31, 2001

(30) Foreign Application Priority Data
Aug. 31, 2000 (JP) 263395-2000
Aug. 31, 2000 (JP) 263505/2000

(51) Int. Cl. C12Q 1/68; G01N 33/543

(52) U.S. Cl. 435/6; 436/518

(57) ABSTRACT

Multiple specimens, typically biological samples having different properties and origins, are bound onto matrix substrates, and oligonucleotides, proteins and drugs are spotted on each matrix in an array to examine those specimens at a time for multiple items.

### BACKGROUND OF THE INVENTION p 1. Field of the Invention

The present invention is directed to examining multiple specimens at a time for multiple items, and provides a method in which matrix substrates with biological samples having different properties and origins bound thereto are prepared, and on each matrix region, oligonucleotides having different sequences, proteins or drugs are spotted in an array, whereby multiple specimens are examined at a time for multiple items.

The present invention also relates to a method in which by using an oligonucleotide having a known base sequence as a detection probe to detect whether a complex is formed by intermolecular bond with this oligonucleotide, detection is made whether or not components having a capability of bonding to the above described detection probe are contained, and a detection substrate having the oligonucleotide as a detection probe fixed on its surface, which is used exclusively for this detecting method.

2. Related Background Art

In identification of partial sequences included in the base sequence of a nucleic acid molecule, detection of a target nucleic acid contained in a sample originated from an organism or identification of genus or species for various bacteria based on the characteristics of the gene DNA of the bacteria, a procedure may be used in which two or more probe DNAs having known base sequences are used to detect whether or not the nucleic acid molecule is a nucleic acid molecule specifically binding to each probe DNA, namely making hybridization with each probe DNA. As an effective approach to performing speedily and accurately examination of the two or more probe DNAs by the hybridization method, a procedure is proposed in which a probe array of two or more probe DNAs arranged regularly on a solid phase is used to detect at a time whether or not the nucleic acid molecule is a nucleic acid molecule specifically binding to each probe DNA.

Among common methods for producing such probe arrays, as described in European Patent No. 373203 (EP 0373203 B1) for example, methods are known in which predetermined nucleic acid probes are synthesized in an array form on a solid phase, and methods in which a plurality of nucleic acid probes synthesized in advance is supplied in an array form on the solid phase.

Prior technical documents disclosing the former methods include, for example, U.S. Pat. No. 5,405,783. Also, as one example of the latter methods, a method in which cDNAs are arranged in an array form on a solid phase using micropipetting is disclosed in, for example, U.S. Pat. No. 5,601,980 and “Science”, Vol. 270, pp. 467, (1995).

The probe array that is prepared with these methods may be an array such that nucleic acid probes are arranged on a solid phase at a high density of 10000 or more probes per square inch. Hybridization reaction with multiple probes are carried out at a time by dipping this high-density probe array into a specimen solution, and in so doing, the base sequence of genes is analyzed based on the base sequence of nucleic acids making hybridization. This method has an advantage that probes are arranged in a high density on a substrate of small area, thereby making it possible to conduct multiple-item examination at a time with a small amount of samples to reduce burden associated with sampling from the subject.

As a method of preparing the high-density probe array for the above described application on the substrate by the DNA synthesis process, a method in which a photolithography technology is applied is disclosed in the aforesaid U.S. Pat. No. 5,405,783, but highly advanced equipment is required for implementing this method, and the method is not easy enough for anyone to use.

Also, in the case where the number of specimens is large but the number of required examination items is not so large, the integration degree of DNA probes on the probe array corresponding to the number of examination items does not need to be very high. Rather, there may be cases where it is necessary to prepare a large number of probe arrays with a small number of desired DNA probes fixed, using a simpler method.

Actually, in the field of clinical examination, there are not necessarily many cases where examinations for more than 10000 items are required. For example, in the case of group health examination and the like, there may be cases where it is more important to examine a large number of specimens with a limited number of items. For examining a large number of specimens in this way, a system is required such that presence of diseases can be speedily examined through comparison with standard samples with respect to each specimen.

In addition, the amount of DNA specimen is generally small as compared to that of oligonucleotide capable of being synthesized and used in the probe. For using it in a normal form in which the probe array substrate is dipped into the specimen solution for hybridization reaction, the amount of specimen DNA allowing the substrate to be dipped sufficiently is required. Therefore, the size of the DNA probe array substrate is limited depending on the amount of specimen DNA, and thus the array needs to be highly dense. Alternatively, as a result of diluting the specimen solution to ensure its volume for the size of the probe array substrate, the concentration of DNA in the specimen solution is reduced, and a procedure is adopted of prolonging reaction time to make compensation for the reduced concentration.

Also, since the amount of sampled specimens is limited inherently because the specimen is an extract from tissues, and it is subjected to pre-processing for making a specimen solution for use in hybridization reaction, specifically extraction of nucleic acid, single-strand formation thereof, and process for labeling, the amount of finally obtained samples is very small. In order to make compensation for that, the sample is subjected to processing for amplification of the amount of DNA such as amplification processing by PCR reaction before it is used for examination and studies. However, there exists a disadvantage that because primers separately prepared are required for carrying out a PCR reaction, such processing can be applied only to specific genes of which primer sequence is known. In addition, there exist sequences that can easily be amplified and sequences that can hardly be amplified in the process of PCR reaction, and thus the efficiency of reaction (rate of amplification) is not uniform. For example, in the case where the content of a specific mRNA in the total amount of extracted mRNA is examined to determine diseases or like based on the content, standard samples providing criteria should be always prepared to make correction on the above described amplification rate.

Although the amount of the specimen solution required for hybridization reaction decreases as the size of the substrate is reduced, there is a limitation on downsizing of the substrate in association with handling. Specifically, it is possible in principle to enhance array density and reduce the number of probes to be placed on the array to downsize the substrate, but if an extremely small substrate is used, a dedicated handling apparatus is required in the process of processing such as hybridization reaction and detection thereafter, which cannot be practical.

Also, for examining cDNA for mRNA that is transcribed with reflection of the process of development of a certain organism, cDNA for mRNA that is transcribed with reflection of each phase in the process of culturing a certain cell, cDNA for mRNA that is transcribed by interaction with drugs, and so on, a DNA array with multiple types of test samples arranged is used. Examples of arraying this test sample are described, for example, in the above described “Science”, Vol. 270, pp 460, (1995). In this case, test samples arrayed on the substrate are dipped using as a probe solution the labeled DNA of known sequence that is derived from genes having a specific function, whereby hybridization reaction is carried out.

If a plurality of items is to be examined at a time using this methodology, DNA probes labeled with different types of fluorescent reagents (fluorochromes) should be prepared depending on the number of items. When detection is made, those different types of fluorescent reagents (fluorochromes) must be detected as distinguished from one another, and therefore their wavelengths and the like should be different as a matter of course. Of course, detection filters corresponding to respective fluorescent reagents (fluorochromes) are also needed for a detector.

This need for simultaneous examination of multiple items for multiple specimens is not characteristic exclusively of hybridization reaction among genes (DNA).

For example, it is also important to examine multiple items with a small amount of samples as to interaction between genes and other substances such as interaction between genes and proteins (DNA binding proteins) and screening of chemicals that are bound to genes. Detection of former DNA binding proteins is used to elucidate the control mechanism of gene expression by proteins such as transcription accelerators, but in the present situation, methods in which DNA fragments are bound to proteins, and thereafter complexes are analyzed by gel electrophoresis are adopted. In this method, the number of specimens that can be analyzed at a time is limited due to usage of gel electrophoresis, and considerable time is required for analysis.

For the field of development of drugs, there may be cases where examination of interaction between genes and administered drugs constitutes an important item in progress of research, but it takes relatively much time and efforts to obtain chemically synthesized products for use in drugs to be researched, and it can be considered that reduction in the amount of drugs to be used in screening results in significant improvement in efficient of their research.

As introduced above, there are cases where when a complex is formed using interaction between two substances such as hybridization between DNAs, formation of a complex of DNA and a protein, and interaction of a drug compound with gene DNA, or the presence or absence of interaction causing a complex to be formed is examined, the amount of samples of one of those two substances is limited, and the limited amount of samples should be used to conduct a series of desired examinations across multiple types as to the presence or absence of formed complexes. That is, development of an examination method in which consumption of samples required for individual examinations can be reduced to carry out examination across multiple types more efficiently within a limited amount of samples is desired.

### SUMMARY OF THE INVENTION

An object of the first invention is to provide a method of examining multiple specimens at a time for multiple items, for example a method in which matrix substrates with biological samples having different properties and origins bound thereto are prepared, and on each matrix region, oligonucleotides or proteins having different sequences and drugs are spotted in an array form, whereby multiple specimens are examined at a time for multiple items.

Another object of the invention is to provide a method in which multiple specimens can also be examined at a time for multiple items in a similar way for interaction between chemicals, especially drugs, and cDNA, binding of proteins to cDNA and the like.

An object of the second invention is to provide a new method in which oligonucleotide of which base sequence is known and which can be obtained relatively easily is used as a detection probe, and when for a limited amount of sampled specimens, the presence or absence of a bonding capability to the above described oligonucleotide as a detection probe or the degree of the bonding capability is examined by the presence or absence of complexes formed between those two substances, or efficiency thereof is evaluated, consumption of specimens required for evaluation for each type of oligonucleotide as a detection probe can be reduced. In addition, the invention also has an object to provide a detection substrate with the above described oligonucleotide being fixed as a detection probe in a predetermined region of its surface, which is used exclusively for the method, and provide a method of preparing the detection substrate.

The examination method of the first invention capable of achieving the above described objects is a method in which a reactivity between a first sample and a plurality of second samples having different properties from one another is examined at a time,

characterized in that in a defined region on a substrate with the first sample bound on the entire surface in advance, the second samples are placed independently of one another as spots having a smaller size than the above described defined region, and then the reactivity between the above described first sample and each of the second samples is tested.

The matrix of biological samples related to the invention that is usefully used for the above examination method is characterized in that two or more types of biological samples of different origins exist in respective matrix regions separated on the substrate.

According to the invention, a substrate with biological samples having different properties and origins (e.g. nucleic acids and proteins) bound in a matrix form in advance can be provided.

There is also provided a method in which DNA probes like oligonucleotides, cDNAs, proteins or chemicals are spotted in an array form on the above described substrate with biological samples having different properties and origins placed in a matrix form to carry out reaction, and the presence or absence of another sample bound to a certain biological sample, the degree of the bonding, and the presence or absence of interaction is examined for multiple items at a time and speedily.

In this method, the area occupied by one specimen is very small because two or more types of specimens are placed on one substrate. Therefore, there is an advantage that the amount of required cDNA may be very small as compared to the case where hybridization reaction is carried out using a conventional DNA array with an enormously large number of DNA probes bound in an array form in advance. Also, there is neither limitation on the size of the DNA array substrate nor inconvenience for handling.

Also, by providing a method in which examination can be carried out even with a small amount of samples, the method opens the door to areas in which examination could not be carried out because conventionally, a sufficient amount of samples cannot be obtained, for example a new examination area in which mRNA obtained from tissues is directly examined.

In addition, according to the invention, a method in which chemicals, proteins and nucleic acids can be examined at a time under the same reaction condition on the same substrate.

A method of detecting object components in test samples according to the second invention is a method in which using as a detection probe oligonucleotide of which base sequence is known, complexes formed between the above described oligonucleotide and the object components are detected to examine whether or not the object components having a capability of binding to the above described oligonucleotide are contained in the liquid test samples, or evaluate the degree of binding capability thereof,

characterized in that there is at least one type of the above described oligonucleotide used as a detection probe, of which base sequence is known,

there are at least two types of test samples to be examined, and

a detection substrate with the above described one or more types of oligonucleotide for detection probes bound to predetermined sections respectively on a predetermined solid substrate is used,

the above described method comprising steps of:

spotting a plurality of predetermined amounts of sample solution for each spot so that a predetermined array shape is formed in the spotted position, for each of the above described two or more types of test samples, in each section with the oligonucleotide for detection probes bound in advance;

detecting the presence or absence of complexes formed between the above described oligonucleotide and the object component, for the above described plurality of spots for each test sample, respectively; and

determining whether or not the object component having a capability of binding to the above described oligonucleotide is contained, or the degree of the capability of binding, based on the result of the above described detection.

Also, the present invention provides a detection substrate that is exclusively used when the above described method of the invention is carried out. That is, the detection substrate of the present invention is a detection substrate with two or more oligonucleotides having known base sequences different from one another fixed on a solid substrate, characterized in that:

the above described plurality of oligonucleotides are bound and fixed in predetermined sections, respectively, so that one type of oligonucleotide exists in each section, and

a plurality of the above described sections in which oligonucleotides are fixed is placed in a matrix form on the surface of the above described solid substrate.

The method of preparing the detection substrate of the present invention is a method suitable for preparation of the above described detection substrate of the invention, and specifically is a method of preparing a detection substrate with two or more oligonucleotides having known base sequences different from one another fixed on a solid substrate, characterized in that:

for the above described solid substrate, a substrate with a plurality of sections separated in a matrix form in advance formed on the surface thereof is used,

the above described a plurality of oligonucleotides is supplied into predetermined sections in predetermined amounts using printing by ink jet process, respectively, so that one oligonucleotide is present in each section, and

the supplied oligonucleotides are fixed in the predetermined sections.

### BRIEF DESCRIPTION OF THE DRAWINGS

FIG. 1 shows one example of an arrangement aspect of defined regions on a substrate in the present invention;

FIGS. 2A and 2B show one example of matrices in the present invention, wherein FIG. 2A is a plan view, and FIG. 2B is a 2B-2B sectional view thereof;

FIG. 3 is a schematic explanatory view of a specimen solution discharging method by bubble jet process that is an embodiment of the present invention;

FIG. 4 is a sectional view of a bubble jet head 105 taken in the 4-4 line in FIG. 3;

FIG. 5 shows a layout of 64 discharged DNA probes on each black matrix;

FIG. 6 shows one example of detection substrates of the present invention, illustrating schematically a situation in which sections in which oligonucleotides being detection probes are fixed are arranged in a matrix form, and a plurality of cDNAs are spotted in a two-dimensional array form onto each section as detection samples;

FIG. 7 illustrates schematically arrangements of respective probes in the detection substrate with 64 DNA probes bound to sections arranged in the form of a 8×8 matrix, respectively;

FIG. 8 shows schematically a pattern of a spot array of total 64×64 in which 64 test samples are spotted in the form of a two-dimensional 8×8 array on each section, for the detection substrate on which sections with probes fixed therein are arranged in the form of the 8×8 matrix;

FIG. 9 shows schematically a result of spotting 64 test samples in the form of the two-dimensional 8×8 array on each section for 64 probes fixed in sections arranged in the form of the 8×8 matrix to carry out hybridization reaction; and

FIG. 10 shows an example of the structure of sections delimited by hydrophobic frame-structured walls provided on the detection substrate of the present invention, and arranged in the form of the 8×8 matrix.

### DETAILED DESCRIPTION OF THE PREFERRED EMBODIMENTS

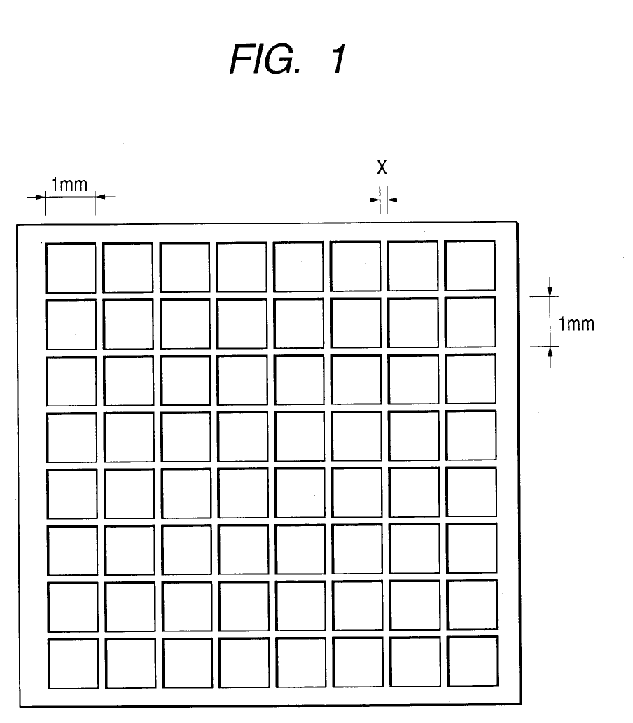

One embodiment of the present invention will be described below referring to FIG. 1. FIG. 1 shows a substrate surface with 64 defined regions formed thereon, wherein each region (matrix) measures 1 mm by 1 mm, and a space x between regions can be selected freely. For methods of preparing biological sample binding matrix substrates, for example, a method can be used in which the solution of a first sample (e.g. biological sample) is printed on the entire surface of defined regions on the substrate as a “solid print pattern” by coating and ink jet processes, or is supplied by methods such as chemical synthesis on the substrate, and is bound in a matrix form on the substrate through adsorption to the substrate or chemical reaction between functional groups existing in the biological sample and functional groups existing on the substrate. Furthermore, the situation in which the first sample is bound on the entire surface of defined regions means a situation in which the first sample is bound across the entire surface such that when a second sample and samples thereafter are supplied in these defined regions, these reactions occur without being limited to the positions in the above described regions in which the samples are supplied. For example, the first sample may be fixed in layered form on the entire surface, or the masses of molecules constituting the first sample may be dispersed on the entire surface in high density with micro-spaces being kept among them.

The defined regions on this substrate may previously be provided on the substrate as a well constituted by sections separated in pattern form by walls of hydrophobic compounds.

Also, when using a substrate with nucleoside acid (cDNA) being a biological sample fixed thereon as the first sample, two or more probe DNAs possibly included in cDNA are contacted with cDNA on the substrate as the second sample and samples thereafter, and products of reaction with the above described probes are detected on the above described solid phase to detect the presence or absence of probe DNA sequences in the above described cDNA, two or more probes are supplied in an array form as mutually independent spots in each matrix with various kinds of CDNA bound in the defined regions, thereby making it possible to perform simultaneous detection with two or more probes.

Also, on the nucleic acid (cDNA) matrix, two or more types of chemicals or proteins that are possibly bound to cDNA are contacted with the probe DNA on the substrate as mutually independent spots, thereby making it possible to perform multiple-item examination composed of these reactions at a time. Multiple-item screening of DNA binding proteins and DNA binding chemicals can be performed at a time by detecting presence of binding of chemicals or proteins to probes on the solid phase.

The present invention is characterized by supplying probe DNA, proteins and chemicals in a form of droplets of small amounts on the matrix on which biological samples such as cDNA are applied, wherein different types of samples are arranged in an array form, thereby making it possible to perform simultaneous multiple-item processing.

Combinations of the first sample fixed in advance on the substrate and the second sample and sample thereafter that are reacted with the first sample may include the following combinations.

Specific examples of the matrix or the like formed of defined regions on the substrate for use in the present invention will be described below.

(Shapes of Matrices with Biological Samples Bound Thereto)

The shapes of matrix patterns are not particularly limited, and may include any shapes, but shapes such as linear, squares and rectangular are preferable in that they can be treated irrespective of how specimens are supplied, in consideration of convenience at the time of supplying specimens on the created substrate. Of course, forms such as circles and ellipses will cause no problems.

Materials that are fixed to the substrate as a first sample may include unknown base sequences derived from organisms, cDNA libraries, mRNA libraries, sets of two or more DNA and RNA, known DNA and RNA synthesized or derived from organisms or sets thereof, chips of cloned oncogenes, protein fractions including at least one type of protein derived from organisms, proteins of single type, mixtures of known proteins of different types, and chemicals.

(Density of Matrices with Biological Samples Bound)

The density of matrices is not particularly limited, but for a preferred form, the density of 400 per centimeter square is preferable. For density of 400/cm², the size of one matrix is a 500 μm square in the case of square form. If samples to be arranged as spots on the array are arranged as spots with diameters of 100 μm, 25 spots are arranged in total with 5 spots high by 5 spots wide. Also, if the diameter of sample solution is 20 μm, the number of spots that can be arranged in a row is 25, and 625 spots can be arranged in total.

(Preparation of a Substrate with Biological Samples Bound Thereto)

Samples originated from organisms (biological samples) include nucleic acids and proteins. Nucleic acids include, for example, mRNA and cDNA, and methods for binding them on the substrate include a method in which nucleic acid extracted and purified in advance is applied to the substrate to fix the nucleic acid by adsorption and electrostatic bond, and a method in which the nucleic acid is fixed by providing covalent bond thorough chemical reaction with functional groups on the substrate using amino groups the nucleic acid has.

The method using negative electric charges of DNA is a method in which nucleic acid is electrostatically bound to a solid carrier subjected to surface treatment with poly positive ions such as polylysine, polyethyleneimine and polyalkylamine, and then blocking of excessive positive ions is carried out, which is generally used.

(Types of Functional Groups of Solid Phases and Nucleic Acids)

Combinations of functional groups that are used for fixation include, for example, a combination of epoxy groups (on solid phase) and amino groups (amino groups in nucleic acid probe terminals or base groups). Methods for introducing epoxy groups to the solid surface include, for example, a method in which polyglycidyl methacrylate having epoxy groups is applied to the solid surface composed of resin, and a method in which a silane coupling agent having epoxy groups is applied to the solid surface made of glass and is reacted with glass.

(Binding of Proteins to the Solid Phase)

Methods of binding proteins to the substrate include methods using adsorption as in the case of nucleic acid and methods using electrostatic binding. Furthermore, methods of forming covalent bond include methods using SH groups of cysteine residues in addition to the above described methods using amino groups.

(Methods of Fixation of Proteins using Thiol Groups)

Methods using cysteine residues for fixation of proteins include, for example, methods using combinations of maleimide groups and thiol groups (—SH). That is, treatment is done so that the solid surface has maleimide groups, whereby thiol groups of cysteine residues supplied to the solid surface can be reacted with maleimide groups of the solid surface to fix proteins.

For methods of introducing maleimide groups to the solid surface, a various kinds of methods may be used, and this can be achieved by, for example, reacting an aminosilane coupling agent with a glass substrate, and then reacting its amino groups with a reagent containing N-(6-maleimidocaproyloxy)succinimide) expressed by the following structural formula (EMCS reagent: manufactured by Dojin Co., Ltd.).

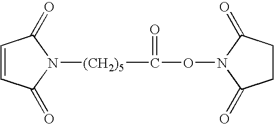

For another example, a reagent containing succinimidyl 4-(maleimidophenyl)butyrate can be used to react with amino groups preferably.

(DNA Matrix Structures Composed of Hydrophobic Matrices)

For an additional form of fixation of biological samples, a method can be used in which a well composed of, for example, hydrophilic and hydrophobic matrices is formed on the solid surface, a structure to prevent coupling among spots is provided in advance, and the DNA prove is supplied in the well to carry out coupling reaction.

(Materials of Matrices/Wells)

When prove solution is put on the separated matrix to carry out coupling reaction, it is preferable that portions constituting the well is hydrophilic, and portions corresponding to the wall surface of the well and the partition between the well and a neighboring well are composed of materials whose surfaces are less compatible with the prove solution. Due to such a treatment, the probe solution can be smoothly supplied to a desired well even if some positional deviation occurs when the prove solution is supplied to the well.

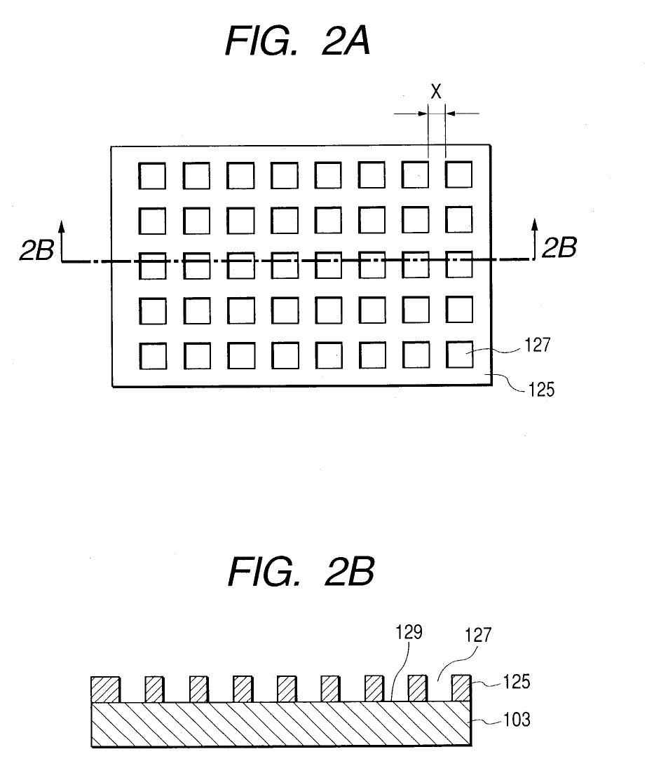

One example of matrices in this embodiment is shown in FIGS. 2A and 2B. FIG. 2A is a plan view, and FIG. 2B is a 2B-2B sectional view thereof. This matrix has a structure in which a matrix patter 125 having a frame structure with formed recesses 127 (wells) placed in the form of a solid phase 103 is provided. The wells 127 separated from one another by the matrix 125 (height) are provided as through-holes (cut-off portions) in the matrix pattern, of which side is constituted by heights and of which bottom 129 has the exposed surface of the solid phase 103. The portion of the exposed surface of the solid phase 103 forms a surface that can be coupled to the probe, and the probe is fixed in a predetermined recess.

Materials forming the matrix pattern include, for example, metals (chrome, aluminum, gold, etc.) and resins. They include resins such as acryl, polycarbonate, polystyrene, polyimide, acrylate monomers and urethane acrylate, and photosensitive resins such as photoresists having black dies and black pigments contained therein. For specific examples of photosensitive resins, UV resists, DEEP-UV resists, ultraviolet cured resins and the like can be used. UV resists may include negative resists such as cyclized polyisoprene-aromatic pisazide resists, phenol resin-aromatic azide compound resists, and positive resists such as novolac resin-diazonaphthoquinone resists.

DEEP-UV resists may include, for example, radiation dispersion type polymer resists such as polymethyl methacrylate, polymethylene sulfone, polyhexafluorobutyl methacrylate, polymethyl isoprobenil ketone and bromo poly 1-trimethylcylilpropine, and dissolution inhibiting resists such as cholate o-nitrobenzyl ester as positive type resists, and may include polovinylphenol-3-3′-diazidediphenylsulfone, and polymethacrylate glycidyl as negative type resists.

Ultraviolet cured resins may include polyester acrylate, epoxy acrylate and urethane diacrylate containing approximately 2 to 10% by weight of one or more types of photopolymerization initiators, which are selected from benzophenone and substituted derivatives thereof, oxime compounds such as benzyl, and so on.

For curbing reflection by the material forming the matrix during detection, light-blocking materials can be effectively used for materials forming the matrix pattern. For this purpose, it is effective to add black pigments in the above described resin, and for black pigments, carbon black and black organic pigments can be used.

Here, if the matrix 125 is composed of resin, the surface of the matrix 125 is hydrophobic. This structure is preferred when aqueous solution is used as a solution containing probes to be supplied to the well. That is, even if the prove solution is supplied to the well, the prove solution is supplied to a desired well quite smoothly. Also, if different probes are supplied among adjacent wells at a time, intermingling (cross contamination) of different probe solutions supplied among these wells can be prevented.

The thickness of the matrix (height from the solid surface) is determined in the light of matrix pattern forming process and the volume of the well, but it is preferably in the range of 1 to 20 μm. Particularly, it can be considered as a thickness range allowing cross contamination to be prevented effectively when the probe solution is supplied to each well though an ink jet process.

(Types of Samples to be Spotted)

Samples to be spotted as droplets onto the above described matrices of biological samples include probe nucleic acids, proteins and chemicals such as drugs.

For probe nucleic acids, in addition to deoxyribonucleic acid, any types of nucleic acids such as ribonucleic acid and peptide nucleic acid may be used as long as they have nucleic acid bases. The length of the oligonucleotide probe is not particularly limited, but it is preferably in the range of 10 mer to 50 mer for carrying out accurate hybridization reaction with cDNA.

For proteins, their own fluorescence can be used to detect DNA bonding proteins.

Some chemicals can also be detected with their own fluorescence.

(Method of Preparing Sample Arrays)

Methods of spotting sample solution on defined positions in the size of several tens to several hundreds of microns include a pin system, an ink jet system and a capillary system.

The pin system refers to a method in which the sample is attached to the pin tip, for example, in such a manner that the pin tip is contacted with the surface of the solution including the sample, and then the tip is mechanically contacted with the solid phase, thereby preparing a sample array. The capillary system using a capillary is such that the sample solution once sucked up to the capillary is mechanically contacted with the solid phase through the tip of the capillary as in the case of the pin system, thereby supplying the sample solution in an array form. For these spotting operations, various apparatuses commercially available from various companies may be used. These methods are considered as most preferable methods in the sense that any sample DNA can be supplied. However, as for quantification, the problem may be unsolved that viscosity varies depending on the length and concentration of DNA. For proteins, these methods are also preferred in the sense that they are deposited independently of the size and viscosity of molecules, but not suitable for quentitative analysis.

(Outline of Sample Array Preparing Methods Through the Ink Jet Process)

Samples capable of being discharged in an ink jet process include chemicals in addition to nucleic acids and proteins.

In the ink jet process, because shearing force is exerted, the length of dischargeable nucleic acids and the size of dischargeable proteins are often limited. However, it is superior in quantification to the pin system and capillary system, and is used more suitably than other systems with respect to discharge of chemicals. Dischargeable nucleic acids are limited to those with relative length to bases of 5 kb or smaller, and dischargeable proteins are limited to those of 1000 K daltons or less. As for chemicals, any chemicals can be discharged.

For liquids for discharge to be used for discharging and supplying samples with ink jets, any liquid can be used as long as it is capable of being discharged from ink jets, and the above described liquid discharged from the head is shot in a predetermined position, and in the state of being mixed with nucleic acid probes and during discharge, the above described nucleic acid probes are not damaged.

And, in terms of dischargeability from the ink jet, particularly from the bubble jet head, for the properties of the above described liquid, it is preferable that its viscosity is in the range of 1 to 15 cps and its surface tension is 30 dyn/cm or larger. Also, if the viscosity is in the range of 1 to 5 cps and the surface tension is in the range of 30 to 50 dyn/cm, the position in which the liquid is spotted on the solid phase is extremely accurate, allowing the method to be used particularly suitably.

Therefore, if the stability of nucleic acid during discharge or the like is taken into consideration, a nucleic acid probe of, for example, 2 to 5000 mer, particularly 2 to 1000 mer is preferably contained in the solution in concentrations of 0.05 to 500 μM, particularly 2 to 50 μM.

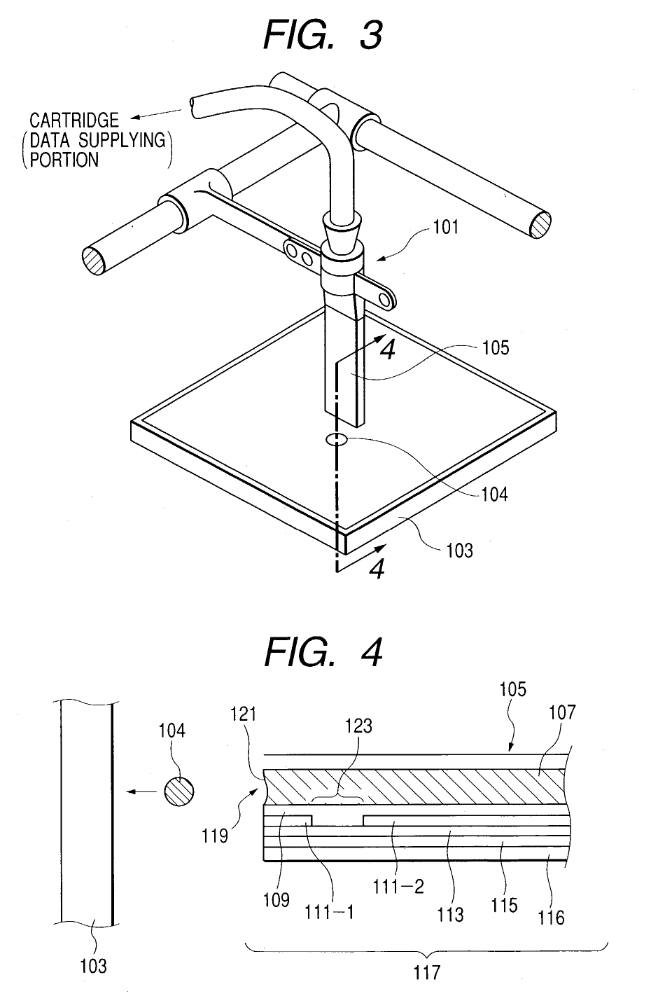

FIG. 3 is a schematic explanatory view of a specimen solution discharging method through the bubble jet process that is one embodiment of the present invention. In FIG. 3, reference numeral 101 denotes a liquid supplying system (nozzle) retaining a solution including a specimen as discharge liquid in such a manner that the solution is capable of being discharged, reference numeral 103 denotes a solid phase having a nucleic probe bound thereto with which the above described specimen is reacted, and reference numeral 105 denotes a bubble jet head having a function of giving heat energy to the above described liquid to discharge it, which is a type of ink jet head. Reference numeral 104 denotes a liquid including the specimen discharged from the bubble jet head. FIG. 4 is a 4-4 line sectional view of the bubble jet head 105 in FIG. 3, and in FIG. 4, reference numeral 105 denotes the bubble jet head, and reference numeral 107 denotes a liquid including a specimen solution to be discharged, and reference numeral 117 denotes a substrate portion having a heat generation portion to give discharge energy to the above described liquid. The substrate portion 117 includes a protective layer 109 formed by silicon oxide and the like, electrodes 111-1 and 111-2 formed by aluminum and the like, an exothermic resistor layer 113 formed by nichrome and the like, a heat storage layer 115, and a support 116 formed by aluminum having good heat-release property.

The liquid 107 including the specimen comes to a discharge orifice (discharge outlet) 119, and forms a meniscus 121 with a predetermined pressure. Here, when electric signals are applied to the electrodes 111-1 and 111-2, a region (foaming region) denoted by reference numeral 123 abruptly releases heat, and the liquid 117 contacted therewith is discharged and flies toward the solid surface 103. The amount of liquid that can be discharged using a bubble jet head having such a structure varies depending on the size of its nozzle, but can be controlled approximately to 4 to 50 picoliters, which is extremely useful as means for placing specimen probes in high density.

And, in terms of dischargeability from the ink jet, particularly from the bubble jet head, for the properties of the above described liquid, it is preferable that its viscosity is in the range of 1 to 15 cps and its surface tension is 30 dyn/cm or larger. Also, if the viscosity is in the range of 1 to 5 cps and the surface tension is in the range of 30 to 50 dyn/cm, the liquid is spotted in an exceedingly accurate position on the solid phase, allowing the method to be used particularly suitably.

Therefore, if the stability of nucleic acid during discharge or the like is taken into consideration, a nucleic acid of, for example, 2 to 5000 mer, particularly 2 to 1000 mer is preferably contained in the solution in concentrations of 0.05 to 500 μM, particularly 2 to 50 μM.

For the composition of discharged liquid, the composition of liquid is not particularly limited, as long as the liquid has no substantial influence on the nucleic acid probe when it is mixed with the nucleic acid probe and when it is discharged from the ink jet, and it can be normally discharged to the solid phase using the ink jet, but preferable are liquids including glycerin, urea, thiodiglycol or ethylene glycol, isopropyl alcohol, and acetyl alcoholene expressed by the following formula.

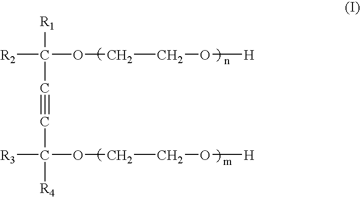

(In the above formula (I), R1, R2, R3 and R4 represent alkyl groups, specifically linear or branched alkyl groups having 1 to 4 carbon atoms, m and n represent integer numbers, respectively, wherein m and n equal 0, or 1≦m+n≦30 holds, and if m+n=1 holds, m or n equals 0).

Further specifically, a liquid containing 5 to 10% by weight (wt %) of urea, 5 to 10 wt % of glycerin, 5 to 10 wt % of thiodiglycol, and 0.02 to 5 wt %, more preferably 0.5 to 1 wt % of acetylene alcohol presented by the above formula (I) is suitably used.

The detecting method of the present invention is a method of detecting a complex formed between oligonucleotide for detection probes and an object component, which is used for the purpose of making evaluation/examination as to whether or not a component having capability of binding to oligonucleotide for use as a detection probe whose base sequence is known, and forming therewith a complex exists in a liquid test sample, and as to the degree of binding capability thereof if such a component exists in the sample. For detecting this complex, oligonucleotide for detection probes is fixed in advance on the solid surface substrate, whereby this fixed oligonucleotide is bound to the object component contained in the test sample, and the formed complex is separated while it is fixed on the solid substrate, and on the basis of a methodology for detecting complexes using proper detecting means, the amount of test samples required at this time is reduced to a very low level, and also, the detection accuracy and sensitiveness are kept at a sufficiently high level.

That is, in this methodology providing a base for the present invention, since surface density of the oligonucleotide for detection probes that is fixed on the solid surface substrate can be kept at a predetermined value, the amount of the formed complex is proportional to the binding capability of the object component, and is also proportional to the concentration of the object component contained in the test sample that is contacted with the solid surface substrate and is made to act on the oligonucleotide. Taking advantage of this characteristic, the test sample is contacted only with the surface with the oligonucleotide for detection probes actually fixed thereon, and the contact area is limited to a certain level, whereby the amount of used test sample is also limited to a certain level. Specifically, by adopting means for spotting a predetermined minimal amount of liquid in the form of droplets, the contact area and the amount of liquid put thereon is controlled with good reproducibility. The amount of the complex that would be automatically fixed on the solid surface substrate with formation is detected for this limited contact area, thereby achieving detection accuracy and sensitiveness essentially as high as those in the case of dipping in the liquid test sample the whole of the solid substrate with oligonucleotide for detection probes fixed thereon.

The complex is detected by the label bound on the surface of the substrate. When a complex of the oligonucleotide and the labeled test sample is formed and the individual spots are sufficiently spaced from each other, detection can be carried out independently for each spot. Therefore, if given or larger spaces are provided between adjacent spots, even though there are spots for different test samples nearby, only spots for desired test samples can be selected to continue detection work without being influenced by those spots. In the detection method of the present invention, in order to satisfy reliably this requirement that given or larger spaces be provided between adjacent spots, a defined array is formed in the spot position as a result of providing predetermined spaces as spaces between spots, and a predetermined amount of sample solution is spotted for each spot to make the spot area (contact area) constant, or make the spot diameter constant to ensure reproducibility because the shape of the spot (contact surface) is generally a circle. As a matter of course, for precluding influence of adjacent spots, a space between spots is selected such that optical signals (fluorescent) and the like derived from the adjacent spots are not mixed in the detection system, in the light of the measured area (diameter in the measurement range) of the detection system selected as appropriate in accordance with the spot diameter. Also, as a matter of course, the detecting method of the present invention really shows its advantages in the case where there exist two or more types of test samples, and they are detected simultaneously.

On the other hand, on the surface to which a plurality of spots of such array forms is provided, one type of oligonucleotide for detection probes should be fixed in uniform surface density. Also, for the section in which the oligonucleotide for detection probes is fixed, its area and shape are selected as appropriate in accordance with the above described array space and the total number of spots to be included in a series of arrays. It is also possible to provide sections having different oligonucleotides fixed therein in different regions on the detection substrate to be used, and place a plurality of sections with two or more oligonucleotides fixed therein, respectively. That is, it can be said that the detecting method of the present invention becomes a more suitable method if used when two or more types of nucleotides are used as detection probes to carry out a series of evaluations simultaneously for a plurality of test samples, with respect to two or more types of object components corresponding to respective oligonucleotides.

Generally, in such an evaluation, it is often the case not that the oligonucleotides for detection probes are predetermined while only an approximate number of test samples to be evaluated is determined. In such a case, it is preferable that as a detection substrate with oligonucleotides for detection probes fixed thereon in advance, a detection substrate with two or more types of detection probes put thereon systematically, having on the substrate surface in a matrix form sections in which respective oligonucleotides are fixed. In this detection substrate with fixed sections arranged thereon in a matrix form, the unit of total number of spots that are made in an array form in each section is fixed, but a plurality of these units of number of spots can be used to carry out evaluation depending on the number of test samples to be actually evaluated, thus enhancing convenience in practice. Furthermore, for the each section arranged in a matrix form, a pattern formed by hydrophobic compounds is preferably provided in its substrateer to provide a form in which mutual regions are separated from one another.

In the detecting method of the present invention, nucleic acid molecules may be selected as object components to apply the same to evaluation as to whether or not they are engaged in double-strand formation into hybrid substances through hybridization reaction with oligonucleotide for detection probes. In this case, the method is an effective method in which evaluation is made at a time even for multiple test samples, as to whether or not nucleic acid molecules including base sequences complementary to known base sequences that oligonucleotide for detection probes has are contained in the test sample. Alternatively, if two or more types of nucleotides for detection probes are provided, and one type of nucleic acid molecules are contained in each test sample, evaluation can be made for the nucleic acid molecule of which base sequences are still unknown, as to whether or not the nucleic acid molecule includes base sequences complementary to known base sequences that each oligonucleotide has, which is effective, for example, for means for making search for a gene group having a set of homologies.

The detection substrate of the present invention is a DNA probe substrate with oligonucleotides for use in probes respectively bound to sections arranged in a matrix form in advance, and particularly for the substrate itself, the bottoms of sections separated by wells (walls) of frame structure matrix patterns formed in advance by hydrophobic compounds are formed as hydrophilic surfaces, thereby making the binding of oligonucleotide easier. Also, by providing this hydrophobic wall, intermingling of DNA probes among adjacent sections can be curbed more reliably.

Also, using these DNA probe substrates, the test sample is spotted in an array form on the matrix of oligonucleotide to carry out hybridization reaction, thereby providing means for checking quickly whether or not nucleic acid molecules having complementarity are included in each test sample for a certain oligonucleotide probe.

In this method, since the number of test samples that are used in hybridization reaction is determined depending solely on the number of spots, the size of the detection substrate is not limited, and by using a substrate of large area, the section in which each probe is fixed can be widened and necessity to enhance density can be eliminated. Thus, since the section in which each probe is fixed can be widened, a wide range of methods can be used such as methods in which a liquid containing probes is applied to, or printed as a “solid printed pattern” through ink jet process on defined regions on the substrate, or methods in which chemical synthesis is carried on the substrate, for means for binding of the probe/oligonucleotide on the substrate.

Also, considering that the probe/oligonucleotide is less expensive and easier to obtain than the test sample, no significant problem arises even if the area of the region to which oligonucleotide is bound is more or less increased, and in this case, with respect to a various kinds of test samples to be spotted, it is not necessary to always spot them in high density. Furthermore, when the test sample is spotted in small amounts, the concentration of the object component that is contained in the test sample is increased, whereby hybridization reaction can be accelerated, thus making it possible to perform high sensitive detection for a short time. In addition, application of the detecting method of the present invention will open the door to fields that could not be considered previously because a sufficient amount of samples could not be obtained, for example a new field in which mRNA obtained from tissues is directly examined.

Furthermore, information of reactivity in association with obtained hybridization reaction is analyzed/evaluated in terms of existence/not existence of complementarity to various kinds of oligonucleotides/probes, with respect to nucleic acid molecules contained in a specific test sample, thereby making it possible to carry out detection having also functions similar to those of conventional DNA arrays (hybridization reaction with multiple probes for one specimen).

Furthermore, the detecting method of the present invention provides means for evaluating as object components the interaction between chemicals, in particular drugs and oligonucleotides, the bonding of proteins to oligonucleotides and the like, and therefore, it can also be used as means making it possible to examine object components included in the test sample for multiple items, with respect to a large number of test samples. In addition, it provides means making it possible to carry out examination on the same substrate at a time and under same conditions even for object components of different properties chemicals, proteins and nucleic acids.

The detecting method of the present invention and the detection substrate for use exclusively therein will be described further in detail below.

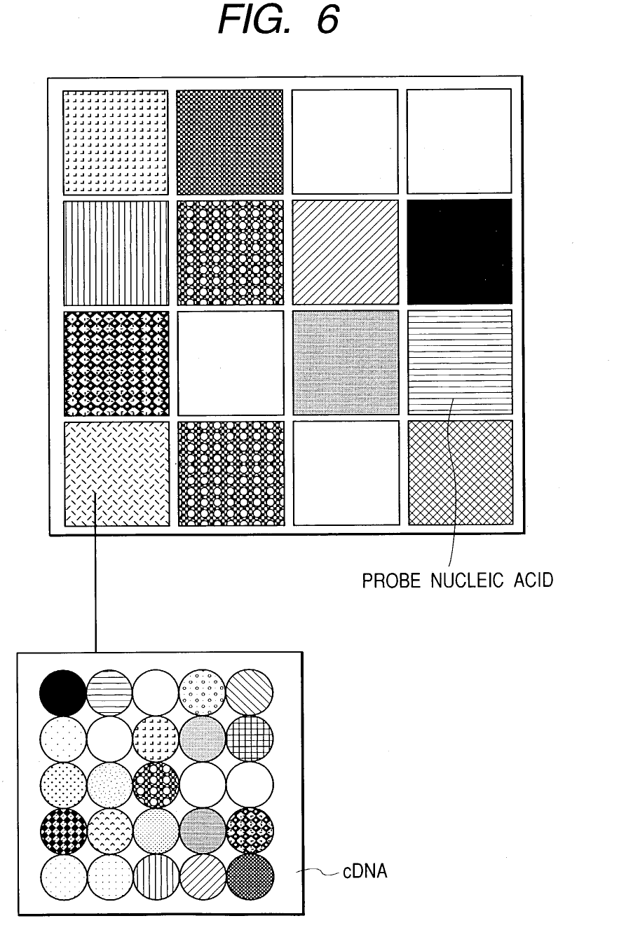

In FIG. 6 is shown an example of applying the detecting method of the present invention to an embodiment in which using cDNA as an object component, a hybrid substance is formed through hybridization reaction with oligonucleotide of known base sequence that is used for detection probes. In the detection substrate shown in FIG. 6, a plurality of rectangular sections separated systematically in a matrix form in advance is provided on the solid surface substrate of rectangular form. The rectangular sections are each spatially isolated by matrix compartments that are surrounding walls. DNA probes that are used for hybridization/probes are each bound uniformly to the bottom surface of the rectangular sections.

Also, attached is an enlarged view showing schematically a situation in which a plurality of test samples including cDNA as an object component, for example two or more types of cDNA solutions prepared based on m-RNAs collected respectively are spotted in the form of the two-dimensional array like a square matrix, in a section with the DNA prove fixed therein.

The detection substrate, the detection probe, components to be detected and the like that are used in the detecting method of the present invention will be described further in detail.

(Oligonucleotides that are used for Detection Probes)

In the detecting method of the present invention, deoxyribonucleic acid can be used for oligonucleotide that is used for detection probes. In addition thereto, ribonucleic acid, peptide nucleic acid and the like can be used. Types thereof are not limited as long as they have desired base sequences, and are capable of being bound to other molecules in those portions, and also as long as they can be fixed on a solid substrate. Also, for portions excluding nucleic acid chains, those modified with non-nucleic acid atom groups and those having additional structures and so on can also be used as long as the above described requirements are satisfied.

Furthermore, for this oligonucleotide that is used for detection probes, a desired amount thereof should be artificially prepared or collected, and its base sequence itself should be known. However, its nucleic acid part should have at least two bases. Its base length is not limited in principle, but if the length exceeds that of 100 bases, difficulty becomes more significant as its base length increases when fixation on the solid substrate is to be carried out, and therefore the base length is preferably restricted to that of 100 bases or less.

For example, when this oligonucleotide is subjected to hybridization reaction with, for example, nucleic acid molecules with the length of more than 100 bases, the length of the oligonucleotide is preferably at least 10 mer for obtaining sufficient bonding. On the other hand, if the length exceeds 50 mer, it is difficult to set conditions for controlling detection of mismatching, thus making it difficult to select and detect only those that are fully matched. Thus, in order to detect mutations, the length is preferably 60 mer or smaller.

Furthermore, the range of 10 mer to 60 mer is a preferable range even when oligonucleotide having desired base sequences, for example DNA is prepared through chemical synthesis.

(Shapes of Sections with Oligonucleotide Fixed Therein, Which is Arranged in a Matrix Form)

The shape of a section itself in which oligonucleotide for detection probes is bound and fixed is not particularly limited. However, if considering that a test samples is spotted in an array form on this section, generally a simpler shape rather than a complicated outside shape is preferably selected. In addition, also when oligonucleotide is bound and fixed, generally a simpler shape is preferably selected for providing uniform surface density in such a section in terms of working efficiency and convenience. Specifically, rectangular forms, for example, line forms, squares and rectangles are preferably adopted. Of course, in principle, forms whose perimeters are formed by curves such as circles and ellipses do not cause any problems.

On the other hand, in the detection substrate of the present invention, when two or more oligonucleotides that are used for detection probes are put on one substrate, sections in which they are fixed are preferably arranged in a matrix form, in terms of working efficiency and convenience. Also, preferably, the form of each section is unified, and its area is also unified.

(Density of Sections Arranged in a Matrix Form)

The density of sections arranged in a matrix form is selected as appropriate depending on the number of oligonucleotides that are put on the detection substrate at a time, but the density of 400 per centimeter square or less is preferable. If the density is 400/cm², and the form of each section is a square, the size of each section is a 500 μm square. If test samples are closely arranged in an array form as spots with diameters of 100 μm, 25 spots are arranged in total with 5 spots high by 5 spots wide. Also, if the diameter of the spot is 20 μm, the number of spots that can be arranged in a row is 25, leading to 625 spots in total. Since the detecting method of the present invention has more significant advantages when there are a large number of test samples and they are examined at a time, the final object of the invention will be more satisfactorily achieved if the density of the section that is arranged is selected so that at least the upper limit of the number of test samples that can be spotted approximately equals the above described value.

For example, when the detecting method of the present invention is applied to test samples including cDNA, the number of test samples to be examined, specifically the total number of types of cDNA often is approximately as many as 3600. In this case, if the diameter of the spot is 100 μm, the size of one section approximately equals a 6 mm square when 60 spots are arranged in rows and columns, respectively. Also, even if the diameter of the spot is 20 μm, the size of one section should be a 1.2 mm square. In this way, in the detection substrate for use in the detecting method of the present invention, there are not a few cases of application objects where the density of sections that are arranged in a matrix form is preferably selected as 400 per centimeter square or less.

Furthermore, in the detecting method of the present invention, the test sample is spotted as droplets, and in the case where the diameter of spot is 100 μm, for example, the amount of liquid required for the droplet of one spot is about 25 picoliters. Even if the number of probes for use in examination is selected as 400 (for example, the number of sections of the matrix to be provided on the substrate is 400) for this spot size, the total amount of liquid required for the whole spots may be no more than 10 nanoliters for each test sample, thus making it possible to carry Out objective examination items with a minimal amount of liquid.

Also, in the conventional method in which the detection substrate is dipped in the solution of the test samples, the amount of required liquid is dependent on the size of the substrate, and thus if the amount of the test sample is essentially very small, the size of the substrate should be reduced in accordance with the amount of liquid, and it is essential to highly integrate probes that are fixed on the substrate. On the other hand, in the detecting method of the present invention, the size of the substrate itself can be freely selected without allowing for the liquid amount the test sample. In addition, when oligonucleotide that is used for detection probes is fixed, the surface density should be uniformed as a matter of course, but it is not necessary to highly integrate a plurality of probes to fix them, thus making the fixing operation easier.

(Fixation of Oligonucleotide on the Substrate)

As means for fixing oligonucleotide that is used for detection probes on the surface of the substrate, a method in which oligonucleotide separately prepared in advance is supplied in predetermined sections by coating or printing to bind the oligonucleotide, or a method in which each oligonucleotide, specifically a DNA probe or the like is synthesized in solid phase on the substrate to prepare originally bound DNA can be used. Furthermore, even in the case where the oligonucleotide is not DNA but, for example, ribonucleic acid or peptide nucleic acid, synthesis on the substrate can be carried out to bind the oligonucleotide as described later.

On the other hand, when oligonucleotide, specifically DNA or ribonucleic acid, peptide nucleic acid or the like separately synthesized or collected in advance is used for detection probes, a process of fixing the oligonucleotide by covalent bond or of fixing it electrostatic coupling on the surface of the substrate can be used.

(Synthesis of Oligonucleotide on the Substrate)

Synthesis of DNA on the substrate includes synthesis on the silicon substrate using photolithography as a methodology disclosed in U.S. Pat. No. 5,445,934. The U.S. Pat. No. 5,445,934 shows a method in which high density DNA probe arrays are prepared by dividing the surface of the silicon substrate into very small areas, and synthesizing DNA for probes. On the other hand, in the detection substrate for use in the detecting method of the present invention, for example, the size of the section in which each probe is fixed may be a 0.5 mm square or larger, and thus it is not always necessary to enhance density. However, also in the detection substrate of the present invention, photodecomposable protective groups, protective groups that are decomposed by chemicals and the like are bound to nucleic acid in advance, and processes of masking, light exposure and reaction are repeated, whereby DNA chains can be synthesized on each section using the methodology described in the U.S. Patent No. 5,445,934 in which four types of nucleic acid bases are bound for each base to stretch the DNA chain having desired base sequences.

(Fixation of Oligonucleotide Synthesized or Collected in Advance)

As means for carrying out fixation using electrostatic coupling, a method in which polylysine, polyethyleneimine and polyalkylamineaone on the solid surface substrate are subjected to blocking using the negative charge of DNA is generally used.

However, in the case of oligonucleotide with base length of 60 or less that is not sufficiently long, the electric charge of its phosphate groups is also weak, and thus binding onto the substrate by the above described method is not necessarily strong. For this oligonucleotide whose base length is not sufficiently long, if a method in which oligonucleotide with functional groups for covalent bond introduced in the terminal of nucleic acid is synthesized in advance, the substrate is subjected to surface processing suitable for functional groups, and the above described functional groups are used to accomplish covalent bond is used, stronger binding can be achieved, which is more preferable.

Also, in the case where the oligonucleotide is RNA, the above described method that is used for DNA may be applied. Alternatively, in the case where the oligonucleotide is peptide nucleic acid, its nucleic acid part may be used to apply the above described method that is used for DNA.

(Types of Functional Groups for use in Fixation by Covalent Bond between the Solid Substrate and Oligonucleotide)

When oligonucleotide is fixed on the solid surface substrate though covalent bond, functional groups are generally introduced in oligonucleotide and the solid surface substrate, respectively, in advance to carry out reaction therebetween. For this combination of functions, a preferable example is a combination such that maleimide groups are introduced in the surface of the substrate and thiol groups (—SH) are introduced in oligonucleotide. Specifically, thiol groups (—SH) are bound to the terminal of oligonucleotide while the solid surface is subjected to processing of forming a coating having maleimide groups, and when oligonucleotide is supplied to the solid surface, the thiol groups (—SH) are made to act on and react with the maleimide groups to perform fixation through formation of covalent bond.

For introducing maleimide groups in the solid surface, various kinds of methods may be used, and for example, an aminosilane coupling agent is reacted with a glass substrate, and then a reagent (EMCS reagent: manufactured by Dojin Co., Ltd.) including N-(6-maleimidocaproyloxy)succinimde expressed by the following formula, whereby a coating layer having maleimide groups can be formed.

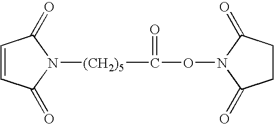

For another example, a reagent containing succinimidyl 4-(maleimidophenyl)butyrate can be used to react with amino groups preferably.

Also, for example, oligonucleotide with thiol groups introduced therein can be synthesized by using 5′-Thiol-Modifier C6 (manufactured by Glen Research Co., Ltd.) as a five prime-end reagent when DNA is synthesized using a DNA automatic synthesizing apparatus. Furthermore, after synthesis, purification processing by high speed liquid chromatography is applied after normal deprotection reaction.

Combinations of functional groups capable of being used for fixation by covalent bond include, for example, a combination of epoxy groups (on the solid surface) and amino groups (the terminal of oligonucleotide) in addition to the above described combination of thiol groups and maleimide groups. Methods for introducing epoxy groups in the solid surface include, for example, a method in which coating is applied to the solid surface constituted by polyglycidyl methacrylate having epoxy groups and a method in which a silane coupling agent having epoxy groups is applied to the solid surface made of glass and is reacted with glass.

(Supply of Oligonucleotide Solution by the Ink Jet Process)

There is no particular limitation on means for supplying predetermined sections on the surface of the solid substrate with a solution containing oligonucleotide to be fixed thereon, as long as a uniform amount of liquid is supplied for each unit area. In the case where printing by the ink jet process and the like is used, a “solid print pattern” is prepared, and then using an ink jet type printer head that is used for ink jet printers, the cartridge for the ink is filled with oligonucleotide solution instead of the ink, and printing for a defined area is carried out. If the amount of liquid to be supplied is small, items of large volume like an ink cartridge are not used, and instead a structure in which a sample supplying portion such as a tube is connected to a head to supply the oligonucleotide solution to the head may be used.

For the oligonucleotide solution for discharge, which is used in this method, a solution that is capable of being discharged in the form of ink jets, and has viscosity suitable for a minimal amount of droplets discharged from the head to be shot onto a desired position is used. In addition, a solvent to be used is selected from solvents that satisfy the above described requirements and give no damages to desired oligonucleotide in the state of being mixed with the desired oligonucleotide and during discharge.

Specifically, in terms of dischageability from the ink jet head, particularly from the bubble jet head, it is preferable that for example the viscosity is in the range of 1 to 15 cps, and the surface tension is 30 dyn/cm or larger as the properties of the solution. In particular, when the viscosity being in the range of 1 to 5 cps and the surface tension being in the range of 30 to 50 dyn/cm are selected, the position in which the solution is shot onto the substrate is extremely accurate, and a supplying method using the bubble jet head is particularly suitably used.

In addition, if stability of oligonucleotide during discharge and the like are considered, the supplying means of the ink jet system is further preferred when for example, a solution containing oligonucleotide of 2 to 100 mer, particularly of 2 to 60 mer in concentrations ranging from 0.05 to 500 μM, preferably from 2 to 50 μM is used.

In applying a discharging method of the ink jet system, the liquid composition of the oligonucleotide solution is not particularly limited, as long as the solution gives practically no damages to desired oligonucleotide in the state of being mixed with the desired oligonucleotide and during discharge as a matter of course as described above, and it can be discharged to the surface of the solid substrate using the ink jet. Furthermore, preferable is a solution containing, for example, glycerin, urea, thiodiglycol or ethylene glycol, isopropyl alcohol, or acetylene alcohol expressed by the following formula in addition to desired oligonucleotide.

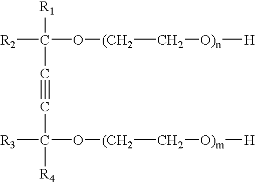

(In the above formula, R1, R2, R3 and R4 represent alkyl groups, for example linear or branched alkyl groups having 1 to 4 carbon atoms, respectively, and m and n represent 0 or positive integer numbers, respectively, and satisfy 1≦m+n≦30). In addition, specifically the liquid composition including 5 to 10 wt % of urea, 5 to 10 wt % of glycerin, 5 to 10 wt % of thioglycol, and 0.02 to 5 wt %, more preferably 0.5 to 1 wt % of acetylene alcohol expressed by the formula (I) allows the discharging method of the ink jet system to be used suitably.

(Structure of Matrices Composed of Hydrophobic Walls and Hydrophilic Wells)

Also, for sections of matrix form that are provided on the solid surface, for example, sections of matrix form constituted by hydrophobic walls (barriers) surrounding hydrophilic wells (recesses) may be formed to prevent coupling between adjacent sections. A structure may also be used in which the solution of oligonucleotide is supplied to the hydrophilic wells (recesses) surrounded by the hydrophobic walls (barriers), and oligonucleotide is fixed only in the bottom of the hydrophilic wells (recesses).

(Materials of Walls/Wells)

When as sections arranged in a matrix form, the solution of oligonucleotide is supplied to the bottom of the wells (recesses) separated by wall (barrier) patterns to carry out binding reaction, it is desirable that the bottom of the wells (recesses) is wetted densely with the solution, but the walls (barriers) have poor wettability with the solution. For example, it is preferable that the solid material constituting the surface of the bottom of the wells (recesses) is much hydrophilic, and the surface of the walls (barriers) and the portion corresponding to partitions with neighboring sections are less hydrophilic. The solution of oligonucleotide supplied in the bottom of the well (recess) is spread across the bottom, but is prevented from finding its way over the wall (barrier) into adjacent sections. Also, even the droplet erroneously supplied in the position related to the wall (barrier) quickly moves into a desired well (recess) having good wettability, and as a result a predetermined amount of oligonucleotide solution can be supplied in the well (recess) more reliably.

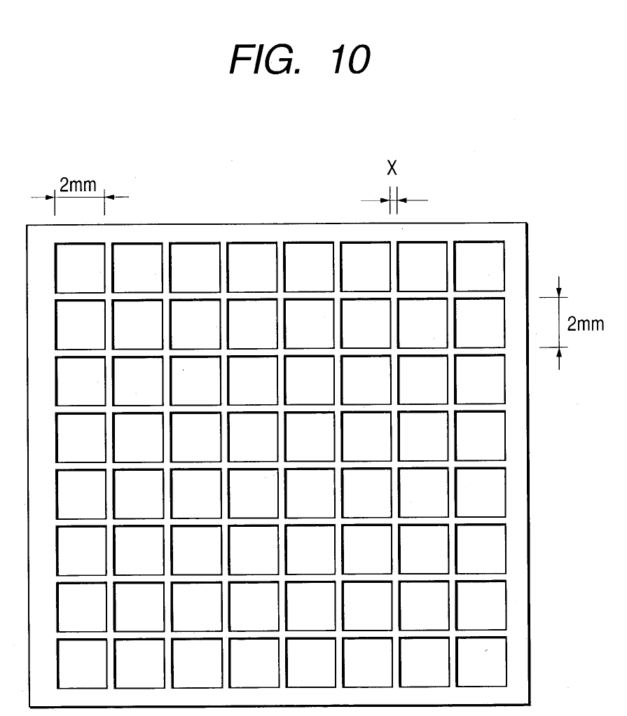

An example of sections arranged in a matrix form that is provided on the detection substrate of the present invention is shown in FIG. 10. The sections in a square matrix form have a structure in which heights (walls) having frame structures are provided on the surface of the solid substrate, and arranged rectangular recesses (wells) are separated. Specifically, the recesses (wells) separated from one another by the heights (walls) having frame structures are formed by coating the entire surface of the solid substrate with a material forming heights (walls), and thereafter providing rectangular through-holes (cut-off portions) to open recesses (wells). Thus, the bottom of the recess (well) has an exposed surface of the solid substrate. The exposed portion of the surface of the solid substrate is subjected to processing for providing a surface to which oligonucleotide can be bound. As a result, oligonucleotide is fixed only in the bottom of this recess (well).

Materials forming heights (walls) having frame structures include, for example, metals (chrome, aluminum, gold, etc.) and resins. Resins include resins such as acryl, polycarbonate, polystyrene, polyimide, acrylate monomers and urethane acrylate, and photosensitive resins such as photoresists having black dies and black pigments contained therein. Furthermore, for specific examples of photosensitive resins, UV resists, DEEP-UV resists, ultraviolet cured resins and the like can be used. UV resists may include negative resists such as cyclized polyisoprene-aromatic pisazide resists, phenol resin-aromatic azide compound resists, and positive resists such as novolac resin-diazonaphthoquinone resists.

DEEP-UV resists may include, for example, radiation dispersion type polymer resists such as polymethyl methacrylate, polymethylene sulfone, polyhexafluorobutyl methacrylate, polymethyl isoprobenil ketone and bromo poly 1-trimethylcylilpropine, and dissolution inhibiting resists such as cholate o-nitrobenzyl ester as positive type resists, and may include borovinylphenol-3-3′-diazidediphenylsulfone, and polymethacrylate glycidyl as negative type resists.

Ultraviolet cured resins may include polyester acrylate, epoxy acrylate and urethane diacrylate containing approximately 2 to 10% by weight of one or more types of photopolymerization initiators, which are selected from benzophenone and substituted derivatives thereof, oxime compounds such as benzyl, and so on.

When detection is carried out using a fluorescent mark, a light-blocking material can be used effectively for curbing light reflex by the material forming this height (wall) having a frame structure. For providing a light-blocking property, it is effective to add black pigments in the above described resins, and in this case, black pigments that can be used may include carbon black and black organic pigments.

Furthermore, if the height (wall) having a frame structure is formed by the above described hydrophobic resin, the surface of the height (wall) is hydrophobic. The configuration in which heights (walls) having frame structures that are formed by hydrophobic materials are provided is more preferable in the case where an aqueous solution is used as a solution containing oligonucleotide to be supplied to the surface of the substrate of recesses (wells). Even if the aqueous solution is supplied in a position related to the surface of the height (wall), it is not persistently attached to the surface of the wall, but gradually moves to the bottom of the recess (well) located in a lower position. Also, solutions of different oligonucleotides are supplied to adjacent recesses (wells), but they are separated from each other by the hydrophobic height (wall), and therefore intermingling (cross contamination) between solutions due to penetration of liquid is prevented.

Furthermore, for the thickness (height from the solid surface) of the height (wall) having a frame structure, the volume of the recess (well) is selected in the light of the amount of the oligonucleotide solution that is supplied to the recess (well), and the thickness is determined as appropriate so that the volume is filled with the solution. Also, depending on methods of forming the height (wall), the thickness is preferable selected such that it is in the range of 1 to 20 μm and satisfies the above described requirement. The thickness of the height (wall) selected in this way is in the range of thickness allowing cross contamination between adjacent wells to be prevented effectively when the oligonucleotide solution is supplied to each well by the ink jet process.

(Types of Specimens)

Object components contained in the test sample to which the detecting method of the invention can be applied include mRNA, cDNA, proteins, cell extracts and chemicals such as drugs.

Furthermore, when cDNA is used as an object component, it is possible to use double-strand cDNA directly, but the single-strand cDNA marked in advance is preferable in forming hybrid substances efficiently and performing detection thereof conveniently.

On the other hand, mRNA is of essentially single-strand, and it is marked in some way to form marked mRNA, thereby making it possible to form hybrid substances efficiently and perform detection thereof. Furthermore, the amount of mRNA in the test sample is generally small, and it is an object component more remarkably reflecting the advantage that the amount of sample solution required for detection can be reduced to a low level, which is characteristic of the detecting method of the present invention. However, since admixture of RAN decomposition enzymes tends to occur during handling, a predetermined amount of substance to curb decomposition of mRNA such as RNA decomposition enzyme inhibitors such as diethyl pyrocarbonate is desirable added in the test sample solution. In addition to mRNA, similarly, the genome of RNA viruses can be an object component. In addition, tRNA, ribosomal RNA and the like can be object components.

On the other hand, when the protein is used as an object component, formed complexes can be detected using the fluorescence emitted by the protein itself.

Also, some chemicals also emit their own fluorescence, and enables formed complexes to be detected using the fluorescence. Chemicals that do not emit fluorescence may be marked by methods using functional groups of compounds. Those to which the detecting method of the invention can be applied may include, for example, chemicals that can be bound to single-strain DNA. In addition, they may include, for example, chemicals that can be bound to single-strain RNA.

(Means for Spotting Test Samples in an Array Form)

In the detecting method of the present invention, the test sample is spotted in an array form in a defined position on the detection substrate. For the purpose of reducing amount of required liquid to a minimal level, the spot diameter is selected so that it is in the range of several tens to 100 μm, but with such a spot diameter, the liquid should be spotted in high uniformity of spotted amounts and high positional accuracy. As means for satisfying this requirement, there are spotting apparatuses of pin systems, ink jet systems and capillary systems.

The pin system refers to a method in which the test sample is attached to the pin tip, and the end point thereof is mechanically contacted with the solid surface, thereby taking out a fixed amount of the test sample. The capillary system using capillaries refers to a method in which the test sample solution is sucked up to the capillary on a temporary basis, and the tip of the capillary is mechanically contacted with the solid surface as in the case of the pin system, thereby taking out a fixed amount of the test sample. A various kinds of spotting apparatuses adopting these two systems are commercially available, and thus commercially available apparatuses may be used.

The spotting apparatuses of the pin system and capillary system enable any types of test samples to be spotted, and are considered as most preferable methods for unknown test samples. For example, however, the viscosity of the test sample solution is varied depending on the length and the concentration of DNA contained in the test sample, and therefore the amount of spotted liquid is varied. Thus, a problem arises in terms of quantification. Also with respect to proteins, the viscosity of the test sample solution is varied depending on the size of the molecules and the concentration, thus raising a problem in terms of quantification.

(Spots in an Array Form of Test Samples by the Ink Jet Process)

Specimens that can be discharged by the ink jet process include chemicals in addition to nucleic acids and proteins.

In the ink jet process, because shearing force is exerted, the length of nucleic acids and the size of proteins that can be discharged are limited. However, it is superior in quantification to the pin system and capillary system, and is used more suitably than other systems, particularly with respect to discharge of chemicals. Preferably, dischargeable nucleic acids are those with relative length to bases of 5 kb or smaller, and dischargeable proteins are those of 1000 K daltons or less. As for chemicals, their molecular weights are generally small enough compared to nucleic acids and proteins, and therefore any chemicals can be discharged except for polymers having extremely large molecular weights.

FIG. 3 illustrates schematically a method of discharging specimen solution by the ink jet process, particularly the bubble jet process, which is one means that is used for spotting test sample solution in the present invention. In FIG. 3. reference numeral 101 denotes a liquid supply system (nozzle) retaining a solution including a specimen as discharge liquid in such a manner that the solution is capable of being discharged, reference numeral 103 denotes a solid phase having a nucleic probe bound thereto with which the specimen is reacted, and reference numeral 105 denotes a bubble jet head having a function of giving heat energy to the liquid to discharge it, which is a type of ink jet head. Reference numeral 104 denotes a liquid (droplet) including the specimen discharged from the bubble jet head. FIG. 4 is a sectional view of the bubble jet head 105 described in FIG. 3. In FIG. 4, reference numeral 107 denotes a liquid including a specimen solution to be discharged from the bubble jet head 105, and reference numeral 118 denotes a substrate portion having a heat generation portion to give discharge energy to the above described liquid. The substrate portion 118 includes a protective layer 109 formed by silicon oxide and the like, electrodes 111-1 and 111-2 formed by aluminum and the like, an exothermic resistor layer 113 formed by nichrome and the like, a heat storage layer 115, and a support 116 formed by aluminum having good heat-release property. The liquid 107 including the specimen comes to a discharge orifice (discharge outlet) 119, and forms a meniscus 121 with a predetermined pressure. In this situation, when electric signals are applied to the electrodes 111-1 and 111-2, a region (foaming region) denoted by reference numeral 123 abruptly releases heat, and the liquid 117 contacted therewith is discharged and flies toward the solid surface 103. The amount of liquid that can be discharged using a bubble jet head having such a structure varies depending on the size of its nozzle, but can be controlled approximately to 4 to 50 picoliters, which is extremely useful as means for arranging probes in high density in a matrix form on the surface of the substrate.

And, in terms of dischargeability from the ink jet, particularly from the bubble jet head, for the properties of the above described liquid, it is preferable that its viscosity is in the range of 1 to 15 cps and its surface tension is 30 dyn/cm or larger. Also, if the viscosity is in the range of 1 to 5 cps and the surface tension is in the range of 30 to 50 dyn/cm, the position in which the droplet is spotted (spot position) on the solid phase is extremely accurate, allowing the method to be used particularly suitably.

In addition, if the stability of nucleic acid during discharge or the like is taken into consideration, single-strain nucleic acid or double-strain nucleic acid of, for example, 2 to 5000 mer, particularly 2 to 10000 mer is preferably contained in the solution. For example, c-DNA chips are preferably contained in the concentration of 0.05 to 500 μM, particularly 2 to 50 μM.

For the composition of discharged liquid, the composition of liquid is not particularly limited, as long as the liquid has no substantial influence on the nucleic acid probe when it is mixed with the nucleic acid probe and when it is discharged from the ink jet, and it can be normally discharged to the solid phase using the ink jet, but preferable are liquids including glycerin, urea, thiodiglycol or ethylene glycol, isopropyl alcohol, and acetyl alcohol expressed by the following formula.

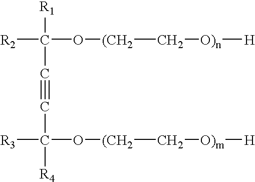

(In the above formula, R1, R2, R3 and R4 represent alkyl groups, specifically linear or branched alkyl groups having 1 to 4 carbon atoms, m and n represent 0 or positive integer numbers, respectively, and satisfy 1≦+n≦30 holds).

Further specifically, a liquid containing 5 to 10% by weight (wt %) of urea, 5 to 10 wt % of glycerin, 5 to 10 wt % of thiodiglycol, and 0.02 to 5 wt %, more preferably 0.5 to 1 wt % of acetylene alcohol is suitably used.

### EXAMPLES

The present invention will be described in detail below using Examples. Furthermore, the Examples shown herein represent one example of most suitable embodiments of the present invention, but the invention should not be limited by these Examples.

### Example 1

A glass substrate with black matrices for specimen matrices for analyzing sequences of p 53 genes on a specimen matrix substrate partitioned by patterns is prepared.

1. Preparation of a Black Matrix Introduction substrate coated with Polylysine.

A glass substrate (60 mm×50 mm) made of synthetic quartz is subjected to supersonic cleaning using 2% sodium hydrate solution, and is then subjected to UV ozone processing to clean the surface. Then, a polylysine solution (manufactured by sigma Co., Ltd.) is applied to the entire surface with a spin coater. In addition, a DEEP-UV resist (negative type resist for black matrices) (BK-739P manufactured by Nippon Steel Chemical Co., Ltd.) is applied thereto with the spin coater so that the thickness after curing is 5 μm, and this substrate is heated for curing at 80° C. for 5 minutes with a hotplate. Using a DEEP-UV aligner, a region of 1 cm×1 cm is proximately exposed to light using a patterned mask so that the distance (X) between adjacent wells in FIG. 1 is 100 μm and the form of the well is a square of 1 mm×1 mm, and then development is carried out with a developing solution of inorganic alkaline solution using a spin drier, and the developing solution is washed out completely with purified water.

Then, the substrate is briefly dried using the spin drier, and is thereafter heated at 180° C. for 30 minutes in a clean oven to have resist fully cured to obtain a substrate in which 400 wells are arranged as a predetermined arrangement and adjacent wells are separated from each other by the black matrix. Furthermore, the volume of each well is calculated as 5 μl if the thickness of the liquid is 5 μm.

2. Fixation of Specimen DNA

(1) Preparation of cDNA Libraries

The p 53 gene is obtained by a PCR reaction from 64 types of cDNA libraries obtained form tumor tissues.

That is, RNA samples were obtained from each tissue collected with biopsies using Catrimox-14 (Biotechnology Co., Ltd.). Based on this sample solution, First-Strand cDNA Synthesis Kit (manufactured by Life Sciences Co., Ltd) is used to obtain cDNA libraries.

(2) Amplification of p53 Genes having T3 Binding Sites by a PCR Method.

Based on the cDNA library, “Human p53 Amplimer set” manufactured by CLONTECH Co., Ltd. is used to carry out PCR reaction.

As a PCR reaction solution, “one shot LA PCR Mix” (Takara Shuzo Co., Ltd.) was used. The composition of the PCR reaction solution is as follows:

| | |
|---|---|
| One shot LA PCR Mix | 25 μl |
| 5′ primer (20 μM) | 1 |
| 3′ primer (20 μM) | 1 |
| cDNA library solution | 1 |
| DW | 22/50 μl. |

The PCR cycle is such that after thermal denaturation at 95° C. for 5 minutes, cycles at 95° C. for 30 seconds, at 55° C. for 30 seconds and at 72° C. for 60 seconds are conducted 29 times, and finally the solution is left for reaction at 72° C. for 5 minutes and is then stored at 4° C.

After the reaction, gel electrophoresis is performed to confirm a product existing in the region of molecular weight of about 300 mer, and purification is carried out with MicroSpin Column S200 (Pharmacia) to obtain p 53 genes (p 53 DNA).

(3) Synthesis of Single-Strain p 53 DNA

Using as a matrix the DNA obtained in the above (2), a single-strain marked DNA is obtained by the PCR reaction using 5′ primer (Takara Shuzo Co., Ltd.). The composition of the reaction solution comprises

| | |
|---|---|
| One shot LA PCR Mix | 25 μl |
| 5′ primer (20 μM) | 1 |
| P 53DNA | 1 |
| DW | 22/50 μl, and |

the reaction cycle is such that cycles at 96° C. for 30 seconds, at 60° C. for 15 seconds and at 60° C. for 4 minutes are repeated 24 times, and finally the solution is stored at 4° C. Thereafter, it is purified with MicroSpin Column S200.

(4) Fixation of p 53 cDNA

5 μl of the single-strain DNA obtained in the above (3) is injected under a microscope into each well of the polylysine-coated substrate with black matrices prepared in the above (1), and is fixed through electrostatic coupling.

3. Analysis of variation of p 53 genes with oligonucleotide probes

The 64 DNAs were selected, focusing attention on the 248th and 249th amino acid sequences of the p 53 gene being a tumor inhibitor gene. That is, it is known that a case of frequent variation in the base sequence of CGGAGG is the case where the first C is changed to T, the second A is changed to G, and the third G of the sequence corresponding to the 249th amino acid is changed to T. Thus, the 64 probes are designed, focusing attention on the base sequence at these three points.

That is, it is a structure in which the total length of the probe is 18 mer, and six bases including this variation are located at the center thereof, and the bases are sandwiched between common sequences. A common sequence corresponds to the range from the five prime-end to the ATGAAC, and the subsequent portion including variation corresponds to the NNGAGN and a further subsequent common portion corresponds to the CCCATC, resulting in a final sequence of 5′ATGAACNNGAGNCCCATC3′. Here, the portion expressed by N corresponds to the A, G, C, and T that are four types of nucleic acid bases. The probe DNA has a sequence complementary to the sequence to be detected (the above described sequence), and thus the sequence thereof is 5′MGGGNCTCNNGTTCAT3′. Rhodamine is coupled to the five prime-end of each probe sequence to mark the prove. Specific base sequences of these 64 types of marked DNA probes are shown in the following Table 1.

TABLE 1

| | |
|---|---|
| SEQ ID NO. | Sequence |

| | |
|---|---|
| 1 | GATGGGACTCAAGTTCAT |
| 2 | GATGGGACTCAGGTTCAT |
| 3 | GATGGGACTCACGTTCAT |
| 4 | GATGGGACTCATGTTCAT |
| 5 | GATGGGAOTCGAGTTCAT |
| 6 | GATGGGACTCGGGTTCAT |
| 7 | GATGGGACTGGCGTTCAT |
| 8 | GATGGGACTCGTGTTCAT |
| 9 | GATGGGACTCCAGTTCAT |
| 10 | GATGGGACTCCGGTTCAT |
| 11 | GATGGGACTCCCGTTCAT |
| 12 | GATGGGACTCCTGTTCAT |
| 13 | GATGGGACTCTAGTTCAT |
| 14 | GATGGGACTCTGGTTCAT |
| 15 | GATGGGACTCTOGTTCAT |
| 16 | GATGGGACTCTTGTTCAT |
| 17 | GATGGGGCTCAAGTTCAT |
| 18 | GATGGGGCTCAGGTTCAT |
| 19 | GATGGGGCTCACGTTCAT |
| 20 | GATGGGGCTCATGTTCAT |
| 21 | GATGGGGCTCGAGTTCAT |
| 22 | GATGGGGCTCGGGTTCAT |
| 23 | GATGGGGCTCGCGTTCAT |
| 24 | GATGGGGCTCGTGTTCAT |
| 25 | GATGGGGCTCCAGTTCAT |
| 26 | GATGGGGCTCCGGTTCAT |
| 27 | GATGGGGCTGCCGTTCAT |
| 28 | GATGGGGCTCCTGTTCAT |
| 29 | GATGGGGCTCTAGTTCAT |
| 30 | GATGGGGCTCTGGTTCAT |
| 31 | GATGGGGCTCTCGTTCAT |
| 32 | GATGGGGCTCTTGTTCAT |
| 33 | GATGGGCCTCAAGTTCAT |
| 34 | GATGGGCOTCAGGTTCAT |
| 35 | GATGGGCCTCAOGTTCAT |
| 36 | GATGGGCCTCATGTTCAT |
| 37 | GATGGGCCTCGAGTTCAT |
| 38 | GATGGGCCTCGGGTTCAT |
| 39 | GATGGGCCTCGCGTTCAT |
| 40 | GATGGGCCTCGTGTTCAT |
| 4T | GATGGGCCTCCAGTTCAT |
| 42 | GATGGGCCTCCGGTTCAT |
| 43 | GATGGGCGTCCCGTTCAT |
| 44 | GATGGGCCTCCTGTTCAT |
| 45 | GATGGGCCTCTAGTTCAT |
| 46 | GATGGGCCTCTGGTTCAT |
| 47 | GATGGGCCTCTCGITCAT |
| 48 | GATGGGCCTCTTGTTCAT |
| 49 | GATGGGTCTCAAGTTCAT |
| 50 | GATGGGTTCTAGGTTCAT |
| 51 | GATGGGTCTCACGTTCAT |
| 52 | GATGGGTCTCATGTTCAT |
| 53 | GATGGGTCTGGAGTTCAT |
| 54 | GATGGGTCTCGGGTTCAT |
| 55 | GATGGGTCTCGCGTTCAT |
| 56 | GATGGGTCTCGTGTTCAT |
| 57 | GATGGGTCTCCAGTTCAT |
| 58 | GATGGGTCTCCGGTTCAT |
| 59 | GATGGGTCTCCCGTTCAT |
| 60 | GATGGGTCTCCTGTTCAT |
| 61 | GATGGGTCTCTAGTTCAT |
| 62 | GATGGGTCTCTGGTTCAT |
| 63 | GATGGGTCTCTCGTTCAT |
| 64 | GATGGGTCTCTTGTTCAT |

Then, for each of the 64 types of marked prove DNAs, a 8 μM solution containing glycerin, urea and thiodiglycol in the final concentration of 7.5%, and acetylenol EH in the final concentration of 1% is prepared. A different probe solution is charged by 100 μl in each of the six nozzles of BJ Printer Head BC 62 (manufactured by Cannon Inc.). Arrangement is made so that six DNAs can be discharged for each head, and two heads are used to discharge 12 DNAs at a time, and the heads are exchanged 6 times to discharge DNAs so that each spot of 64 DNA is formed independently. In this way, total 64 probes are discharged in the form of the 8×8 array in each well of black matrix coated with polylysine.

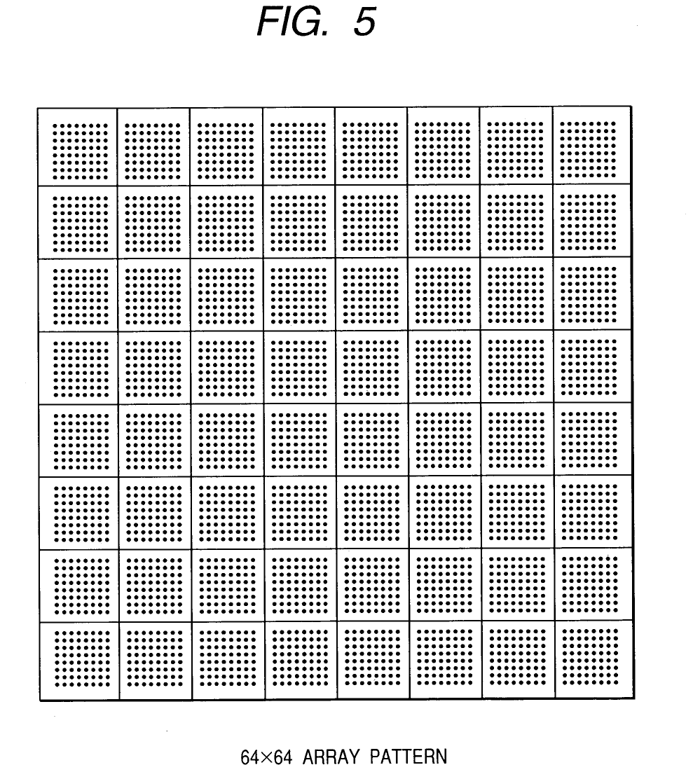

FIG. 5 shows an arrangement on each black matrix of 64 DNA probes that are discharged. In this case, 64 DNA probes are spotted in one matrix.

Thereafter, this substrate in which each probe is spotted is left in a humidifier chamber set at 40° C. to carry out a hybridization reaction.

Thereafter, the substrate is cleaned with a 10 mM phosphate buffer containing 100 mM NaCl to remove DNA probes that have not been engaged in the formation of the hybrid substance.

DNA arrays after the hybridization reaction are observed using an inverted fluorescence microscope equipped with a filter set suitable for rhodamine.

If the gene as a specimen has normal base sequences, spots of highest fluorescence intensity should be observed in the gene at the location of the relative 42th DNA prove. It can be considered that those are derived from the hybrid of the p 53 gene having normal sequences amplified with the probe DNA and PCR. In a varied gene, detectable spots are observed at the location other than the 42th location, and a varied sequence can be known from the DNA probe supplied to the location.

### Example 2

(Evaluation of Existence/Not Existence of Carcinogenic Genes using mRNA)

1. Extraction of mRNA

“QuickPrep Micro mRNA Purification Kit” (manufactured by Amersham Pharmacia biotech co., Ltd.) is used to extract mRNA from tumor tissues collected with the biopsy. This mRNA is bound to a polylysine substrate with black matrices as in the case of Example 1.

2. Examination of Existence/Not Existence of Carcinogenic Genes and the Type Thereof with Various Kinds of Carcinogenic Gene Probe Arrays.

Sets of cloned oncogenes (18 types, manufactured by Takara Shuzo Co., Ltd.) are purchased, and then “LabelITnon-RI Labeling Kits” are used to perform rhodamine marking.

18 types of marked oncogene probes are spotted as an arrangement of 4×5 on the above described substrate with mRNA bound thereto, using a microarray preparing apparatus (pin system) manufactured by Cartesian Technologies Co., Ltd.

Further, a hybridization reaction is carried out as in the case of Example 1.

The type of oncogenes existing in the mRNA section extracted from each tissue can be known.

At this time, detection can be sufficiently performed with one type of marks irrespective of types of oncogenes.

The second invention will be describe more specifically with Examples below.

### Example 3

An example of procedures for preparing an substrate with oligonucleotide bound thereto will be described below. In this embodiment, a detection substrate with oligonucleotide bound to a region of 2 mm square on a glass substrate was prepared in accordance with the procedure described below.

1. Cleaning of the Substrate

A glass substrate of 1-inch square was placed in a rack, and was soaked in a detergent for ultrasonic cleaning. Thereafter, it was subjected to ultrasonic cleaning in the above described detergent for 20 minutes, followed by removing the detergent by rinsing. Furthermore, it was rinsed with distilled water, followed by further performing ultrasonication for 20 minutes in a container containing distilled water.

Then, this glass substrate was soaked for 10 minutes in 1N sodium hydrate solution heated in advance. After it was taken out from the solution, the 1N sodium hydrate solution adhered to the surface was washed out with water, and thereafter cleaning with distilled water was continued.

2. Surface Treatment

The above described cleaned glass substrate was soaked in an aqueous solution of 1% silane coupling agent (manufactured by Shin-Etsu Chemical Co., Ltd., Trade name: KBM 603) at room temperature for 20 minutes, followed by spraying nitrogen gas on the both sides of the substrate to drive off water for drying. The substrate was baked for one hour by using an oven heated to 120° C. to complete treatment of the surface of the glass substrate with a silane coupling agent.

On the other hand, 2.7 mg of EMCS (N-(6-Maleimidocaproyloxy)sucinimide: manufactured by Dojin Co., Ltd.) was weighed, and was dissolved in a solution of DMSO/ethanol (1:1) (final concentration of 0.3 mg/ml). The glass substrate subjected to the treatment with a silane coupling agent was soaked in this EMCS solution for two hours to carry out the reaction between the amino group of the silane coupling agent covering the surface of the substrate and the succinimide group in the EMCS solution. In association with this reaction, the substrate is covered with EMCS through the silane coupling agent. In the obtained glass surface, a maleimide group derived from the EMCS exists on the surface. The glass substrate taken out after the reaction with the EMCS solution is cleaned with distilled water, and is thereafter dried with nitrogen gas. This glass substrate subjected to the surface treatment for introducing a maleimide group will be used for a binding reaction with DNA described later.

3. Synthesis of DNA for Fixing Glass Substrates

Oligonucleotide having a base sequence of the following Sequence 1 is chemically synthesized for fixation on the glass substrate. This sequence 1 is a 18 mer sequence including in its central part a base sequence with a base length of 6 to code 248th and 249th amino acids in an amino acid sequence of a gene product (peptide chain) that is coded by the p 53 gene known as a tumor suppressor gene. Also, A SH group is introduced in its 5′ end for fixation on the glass substrate.

5′ HS-GATGGGCCTCCGGTTCAT3′ Sequence 1

The SH group is introduced by using a commercially available reagent Thiol-Modifier (manufactured by GlenResearch Co., Ltd.) on a DNA automatic synthesizing apparatus. Subsequently, normal deprotction was carried out to recover DNA, and the DNA was purified by high speed liquid chromatography, and was then used in the following processes.

4. Discharging of DNA Using a BJ Printer Head and Binding Thereof to a Substrate

The above described synthetic oligonucleotide (DNA) was dissolved in water, and the solution was diluted to the a concentration of 8 μM using SG Clear (a solution containing 7.5% of glycerin, 7.5% of urea, 7.5% of thiodiglycol and 1% of acetylenol EH).

100 μl of this oligonucleotide solution was charged into the nozzle of BJ printer head BC 62 (manufactured by Canon. Inc.) with the nozzle modified so that it is suitable for a small amount of samples (discharged amount). This modified printer head was set in a plotting apparatus to perform printing over the surface of the glass substrate as an area of “solid print” of 2 mm square with the oligonucleotide solution. Furthermore, the modified printer head that was used is used for bubble jet type ink jet printing and enables printing to be performed at a resolution of 360×720 dpi.

Thereafter, the glass substrate coated with the oligonucleotide solution was left in a humidifier chamber for 30 minutes to carry out a reaction between the maleimide group on the surface of the substrate and the thiol group (HS—) of oligonucleotide. Thereafter, unreacted oligonucleotide was removed. The prepared substrate to detect is a substrate with the synthetic DNA (oligonucleotide) of the above described Sequence 1 bound to a predetermined section of 2 mm square on the glass substrate through covalent bond.

### Example 4

Supply of cDNA Solution to the Surface of the Substrate with Oligonucleotide Bound Thereto and Hybridization Reaction.

From various kinds of cDNA libraries obtained from tumor tissues, p 53 gene fragments were PCR-amplified, and then only one side chains were reamplified using primers marked in advance to prepare marked single-strain cDNA for use as test samples. The hybridization reaction was carried out between this marked single strain DNA derived from the p 53 gene and the DNA probe bound on the detection substrate prepared in Example 3.

1. Preparation of Test Samples

From 64 types of cDNA libraries obtained from tumor tissues, p 53 gene fragments were obtained by the PCR reaction.

Specifically, first, all RNA samples were separated/collected from respective tissues collected with the biopsy, using Catrimox-14 (Biotechnology Co., Ltd.). On the basis of the all RNA sample solutions, a c-DNA library was prepared using First-Strand cDNA Synthesis Kit (manufactured by Life Science Co., Ltd.). A primer for amplifying p 53 genes was added to this CDNA library to amplify P 53 gene fragments. With this PCR amplification product as a template, the marked five side primer was used to carry out the PCR reaction (DNA synthetic reaction) to amplify only one side chains. By this amplification, marked single strain DNA derived from the p 53 gene can be prepared.

(1) Amplification of p 53 Gene Fragments having a T3 Binding Site in the Terminal by the PCR Method.

For using a primer for auto sequencers (Takara Shuzo Co., Ltd) using T3 promoters as the above described marked primer, a primer having a T3 site in the terminal and having coupled to its downstream a base sequence allowing the p 53 gene part to be amplified was first synthesized. The PCR reaction was carried out using this primer to obtain a PCR amplification product having a T3 promoter site coupled to the p 53 gene part.

In this example, for the five prime-end primer for amplifying p 53 genes, the primer with a base sequence having a T3 promoter site coupled to its five side (T3-P53) was prepared. The base sequence is shown below.

5′AATTAACCCTCACTAAAGGGAACCTGAGGTTGGCTCTGACTGTACCACCATCC3′

In the sequence, the underlined part on the side of five prime-end represents a T3 polymerase binding site. On the other hand, for a three prime-end primer for amplification, a three prime-end primer attached in a commercially available amplification kit, “Human p 53 Amplimer Set” of CLONTECH Co., Ltd. was used. For a PCR reactive solution, “one shot LA PCR Mix” (Takara Shuzo Co., Ltd.) was used.

The solution composition in the PCR reaction has:

| | |
|---|---|
| one shot LA PCR Mix | 25 μl |
| T3-P53 primer (20 μM) | 1 μl |
| 3′ primer (20 μM) | 1 μl |
| cDNA library solution | 1 μl |
| DW | 22 μ1/50 μl, |

and for the PCR cycle, a condition of conducting cycles at 95° C. for 30 seconds, at 55° C. for 30 seconds and at 72° C. for 60 seconds at 29 times after thermal denaturation at 95° C. for 5 minutes and finally keeping the solution at 72° C. for five minutes was used, and the reactant was stored at 4° C. on a temporary basis after it was cooled.

After the reaction, gel electrophoresis was carried out to confirm a PCR product existing in the region of molecular weight of about 300 mer. This PCR product was purified with Micro Spin Column S200 (Pharmacia) to obtain p 53 gene fragments to which the T3 primer can be coupled (T3-linked p 53 DNA).

(2) Synthesis of Marked Single Strain DNA using Labeled T3 Primers (Rho-T3).

With the p 53 gene fragment obtained in (1) as a matrix, single strain marked DNA was obtained with the PCR reaction, using a Rho-T3 primer (Takara Shuzo Co., Ltd.). The composition of the reactive solution had:

| | |
|---|---|
| one shot LA PCR Mix | 25 μl |
| Rho-T3primer (10 μM) | 1 μl |
| T3-linked p 53 DNA | 1 μl |
| DW | 23 μl/50 μl, |

and for the reaction cycle, a condition of conducting cycles at 96° C. for 30 seconds, at 50° C. for 15 seconds and at 60° C. for 4 minutes 24 times was used, and the reactant was stored at 4° C. on a temporary basis after it was cooled. It was purified with Micro Spin Column S200, and thereafter gel electrophoresis was carried out to confirm desired rhodamine labeled single strain DNA synthesized through the PCR reaction.

2. Supply of Test Sample Solution

Sodium chloride was added in the test sample obtained in the above described process, namely the solution of rhodamine marked single strain DNA derived from the p 53 gene so that the final concentration of the solution was 1M. The solution of rhodamine marked single strain DNA derived from the p 53 gene, which had been prepared from 64 types of c DNA libraries was injected into each well of a 96-hole microtiter plate. These solutions of rhodamine marked single strain DNA were spotted as an arrangement of 8×8 onto the detection glass substrate with the DNA probe of Sequence 1 obtained in Example 3 in the form of 2 mm square, using a microarray preparing apparatus (pin system) manufactured by Cartesian Technologies. The diameter of each spot was 100 μm.

3. Hybridization Reaction.

This detection substrate with total 64 types of rhodamine marked single strain DNA solutions being sample specimens spotted thereon was left in a humidifier chamber set at 40° C. to carry out a hybridization reaction for 3 hours. Thereafter, the detection substrate was washed with a 10 mM phosphate buffer containing 100 mM NaCl to remove test samples that had not been engaged in the formation of hybrid substances.

After the hybridization reaction, the test sample spotted in the form of a two-dimensional array of 8×8 was observed using an inverted fluorescence microscope equipped with a filter set for excitation light and fluorescence suitable for fluorescence marked rhodamine. For the most part of the spots, red fluorescence derived from fluorescence marked rhodamine in association with the formation of hybrid substances was observed. However, fluorescence intensity was weak for six spots and no fluorescence was observed for one spot.

For this, it can be considered that since in the p 53 gene derived from corresponding six types of tumor cells, variation occurs somewhere in the base sequence corresponding to the 248th and 249th of the amino acid sequence of the p 53 gene product (p 53 protein), the amount of formed hybrid substances is small due to its mismatch, and in association therewith, the fluorescence intensity from the fluorescence mark is weak. For the test sample in which fluorescence was not observed, it can be considered from the fact that hybrid substances were not formed that in p 53 cDNA fragments contained in the sample, deficiency occurs in the base sequence to code the above described 248th and 249th of the amino acid sequence, and consequently hybrid substances could not be formed.

### Example 5

Preparation of Array Form Spots of Test Samples on the Probe Matrix Detection Substrate with Multiple Oligonucleotides Fixed Thereon.

1. Preparation of 64 Probe Matrices.

Processing was performed as in the case of Example 3 to prepare a glass substrate having a maleimide group. 64 DNA of which base sequences are shown in Table 2 were printed (applied) thereon in the area of 2 mm square, respectively, using a bubble jet printer head similar to that of Example 3 to prepare a detection substrate on which sections with 64 types of prove DNAs fixed therein were arranged in a matrix form.

Focusing attention on the 248th and 249th amino acids of the amino acid sequence of the gene product (p 53 protein) of the p 53 gene being a tumor suppressor gene, 64 DNAs of which base sequences are shown in Table 1 were selected on the basis of the base sequence to code these two amino acids so that a sequence with various kinds of base variations added thereto was obtained. Specifically, it is known that a case of frequent variation in the base sequence CGGAGG providing a base is the case where the first C of the CGG to code the 248th amino acid is changed to T, the second A is changed to G, and the third G of the AGG to code the 248th amino acid is changed to T. Thus, 64 probes were designed to provide sequences capable of being bound to base sequences with these bases at three positions varied in various kinds of forms.

Actually, it was a structure in which the total length of the probe was 18 mer, six bases including this variation were located in the center thereof, and common base sequences with base lengths of 6 were placed before and after the six bases. More specifically, the structure has a common sequence of ATGAAC from the side of the five prime-end, the base sequence of NNGAGN as a portion including the variation, and a common sequence of CCCATC on the side of three prime-end.

It was a base sequence a base sequence complimentary to the sequence of 5′ATGAACNNGAGNCCCATC3′. That is, it was a probe expressed by 5′GATGGGNCTCNNGTTCAT3′. Furthermore, since it is a DNA probe, the portion denoted by N in the above described base sequence refers to any one of A, G, C and T that are four DNA nucleic acid bases.

TABLE 2

| | |
|---|---|
| 1 | 5′-GATGGGACTCAAGTTCAT-3′ |
| 2 | 5′-GATGGGACTCAGGTTCAT-3′ |
| 3 | 5′-GATGGGACTCACGTTCAT-3′ |
| 4 | 5′-GATGGGACTCATGTTCAT-3′ |
| 5 | 5′-GATGGGACTCGAGTTCAT-3′ |
| 6 | 5′-GATGGGACTCGGGTTCAT-3′ |
| 7 | 5′-GATGGGACTCGCGTTCAT-3′ |
| 8 | 5′-GATGGGACTCGTGTTCAT-3′ |
| 9 | 5′-GATGGGACTCCAGTTCAT-3′ |
| 10 | 5′-GATGGGACTCCGGTTCAT-3′ |
| 11 | 5′-GATGGGACTCCCGTTCAT-3′ |
| 12 | 5′-GATGGGACTCCTGTTCAT-3′ |
| 13 | 5′-GATGGGACTCTAGTTCAT-3′ |
| 14 | 5′-GATGGGACTCTGGTTCAT-3′ |
| 15 | 5′-GATGGGACTCTCGTTCAT-3′ |
| 16 | 5′-GATGGGACTCTTGTTCAT-3′ |
| 17 | 5′-GATGGGGCTCAAGTTCAT-3′ |
| 18 | 5′-GATGGGGCTCAGGTTCAT-3′ |
| 19 | 5′-GATGGGGCTCACGTTCAT-3′ |
| 20 | 5′-GATGGGGCTCATGTTCAT-3′ |
| 21 | 5′-GATGGGGCTCGAGTTCAT-3′ |
| 22 | 5′-GATGGGGCTCGGGTTCAT-3′ |
| 23 | 5′-GATGGGGCTCGCGTTCAT-3′ |
| 24 | 5′-GATGGGGCTCGTGTTCAT-3′ |
| 25 | 5′-GATGGGGCTCCAGTTCAT-3′ |
| 26 | 5′-GATGGGGCTCCGGTTCAT-3′ |
| 27 | 5′-GATGGGGCTCCCGTTCAT-3′ |
| 28 | 5′-GATGGGGCTCCTGTTCAT-3′ |
| 29 | 5′-GATGGGGCTCTAGTTCAT-3′ |
| 30 | 5′-GATGGGGCTCTGGTTCAT-3′ |
| 31 | 5′-GATGGGGCTCTCGTTCAT-3′ |
| 32 | 5′-GATGGGGCTCTCGTTCAT-3′ |
| 33 | 5′-GATGGGCCTCAAGTTCAT-3′ |
| 34 | 5′-GATGGGCCTCAGGTTCAT-3′ |
| 35 | 5′-GATGGGCCTCACGTTCAT-3′ |
| 36 | 5′-GATGGGCCTCATGTTGAT-3′ |
| 37 | 5′-GATGGGCCTCGAGTTCAT-3′ |
| 38 | 5′-GATGGGCCTCGGGTTCAT-3′ |
| 39 | 5′-GATGGGCCTCGCGTTCAT-3′ |
| 40 | 5′-GATGGGCCTCGTGTTCAT-3′ |
| 41 | 5′-GATGGGCCTCCAGTTCAT-3′ |
| 42 | 5′-GATGGGCCTCCGGTTCAT-3′ |
| 43 | 5′-GATGGGCCTCCCGTTGAT-3′ |
| 44 | 5′-GATGGGCCTCCTGTTCAT-3′ |
| 45 | 5′-GATGGGCCTCTAGTTCAT-3′ |
| 46 | 5′-GATGGGCCTCTGGTTCAT-3′ |
| 47 | 5′-GATGGGCCTCTCGTTCAT-3′ |
| 48 | 5′-GATGGGCCTCTTGTTCAT-3′ |
| 49 | 5′-GATGGGTCTCAAGTTCAT-3′ |
| 50 | 5′-GATGGGTTCTAGGTTCAT-3′ |
| 51 | 5′-GATGGGTCTCACGTTCAT-3′ |
| 52 | 5′-GATGGGTCTCATGTTCAT-3′ |
| 53 | 5′-GATGGGTCTCGAGTTCAT-3′ |
| 55 | 5′-GATGGGTCTCGGGTTCAT-3′ |
| 56 | 5′-GATGGGTCTCGCGTTCAT-3′ |
| 56 | 5′-GATGGGTCTCGTGTTCAT-3′ |
| 57 | 5′-GATGGGTCTCCAGTTCAT-3′ |
| 58 | 5′-GATGGGTCTCCGGTTCAT-3′ |
| 59 | 5′-GATGGGTCTCCCGTTCAT-3′ |
| 60 | 5′-GATGGGTCTCCTGTTCAT-3′ |
| 61 | 5′-GATGGGTCTCTAGTTCAT-3′ |
| 62 | 5′-GATGGGTCTCTGGTTCAT-3′ |
| 63 | 5′-GATGGGTCTCTCGTTCAT-3′ |
| 64 | 5′-GATGGGTCTCTTGTTCAT-3′ |

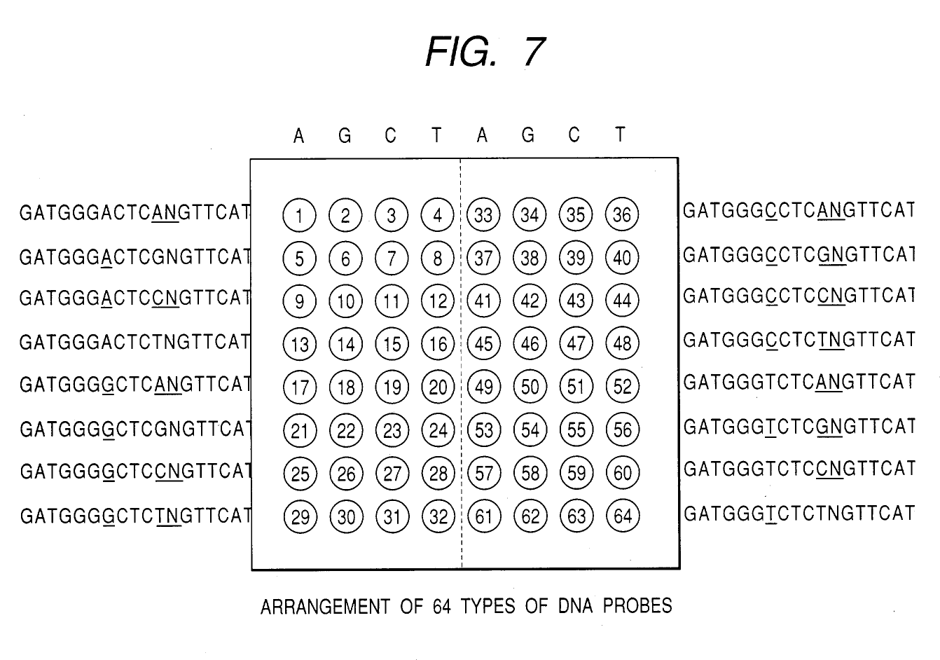

Then, for each of the 64 types of labeled prove DNAs, a 8 μM solution containing glycerin, urea and thiodiglycol in the final concentration of 7.5%, respectively, and acetylenol EH in the final concentration of 1% was prepared. As in the case of Example 4, using BJ Printer Head BC 62 (manufactured by Cannon Inc), different DNA probe solution was charged by 100 μl in each of the six nozzles of the printer head, and using a plurality of such printer heads, a detection substrate with total 64 DNA probes applied to and fixed in each section of 2 mm square in the form of “solid print” and arranged in a matrix form (8×8) was prepared. A schematic layout of the 64 DNA probes arranged in a matrix form (8×8) on the detection substrate is shown in FIG. 7.

2. Preparation of Array Spots of Test Samples.

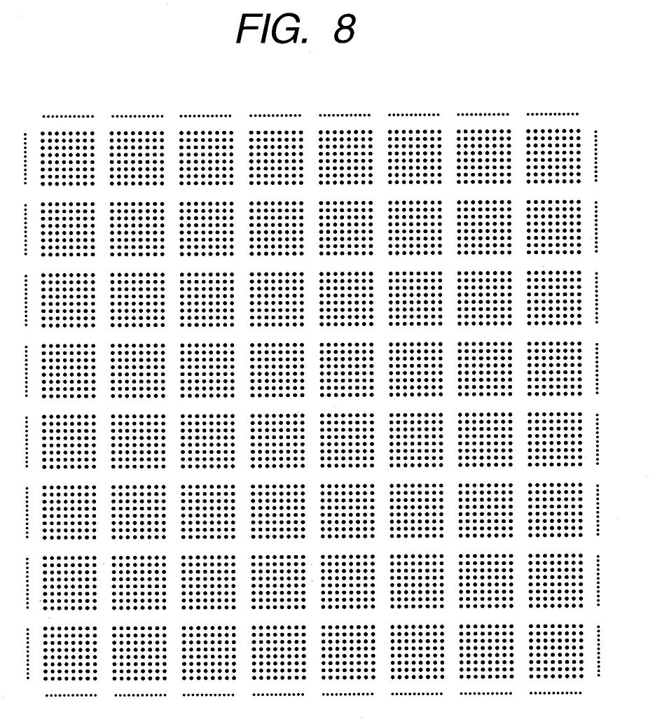

As in the case of Example 4, 64 types of labeled cDNAs were spotted in the form of the two dimensional 8×8 array on each region of 2 mm square for fixing probes. Specifically, as schematically shown in FIG. 8, a pin system array preparing apparatus was used to form spots in the form of the two dimensional 8×8 array on the sections arranged in a matrix form (8×8) in which each DNA probe was fixed.

3. Hybridization Reaction.

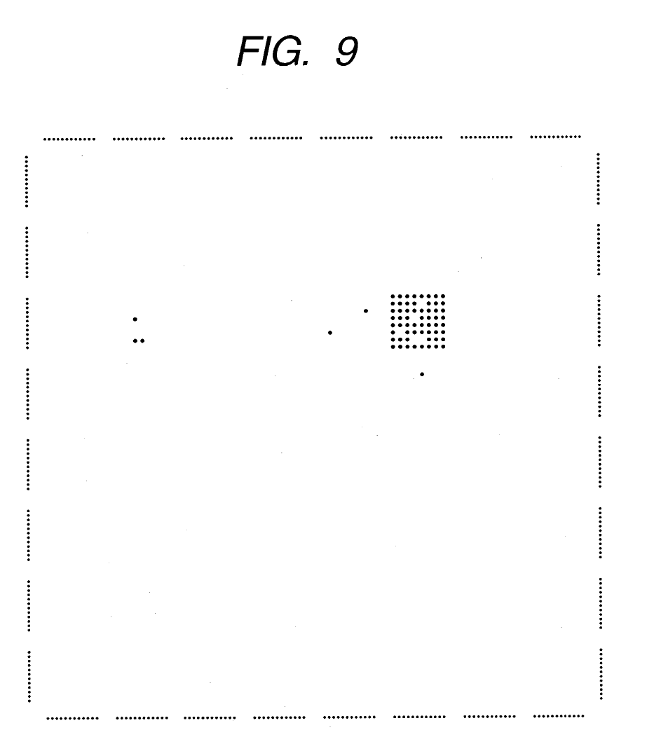

A hybridization reaction was carried out using conditions and procedures similar to those of Example 4. The result thereof is shown in FIG. 9. In the arrangement shown in FIG. 7, with respect to spots on probes corresponding to the base sequence of the 42nd normal gene, fluorescence intensity was weak for six spots as in the case of Example 4. Also, no fluorescence was observed for one spot. In addition thereto, it was observed that fluorescent was emitted from the spot at three points in the tenth probe region, at two points in the 41st probe region, and at one point in the 46th probe region, respectively.

Spot positions in which fluorescence in association with the formation of hybrid substances was observed in the prove region having these base sequences including variations corresponded to spot positions of weak fluorescence intensity in the probe region having the above described 42nd original base sequence. Thus, if the base sequences of the probes are compared between both the regions, the base sequence of the tenth probe is ACTCCG, the base sequence of the 41st probe is the CCTCCA, and the base sequence of the 46th probe is CCTCTG with respect to the original base sequence of CCTCCG that the 42nd probe has. For their complementary sequences, it can be understood that with respect to the CGGAGG in the 42nd probe, the CGGAGT and G were changed to T in the tenth probe, the TGGAGG and C were changed to T in the 41st probe, and the CAGAGG and G were changed to A in the 46th probe. That is, it was confirmed that in test samples forming hybrid substances with these tenth, 41st and 46th probes, CDNA fragments contained therein derived from the p53 gene caused one base mismatch with respect to the 42nd probe due to the above described variations.

By this method, existence/not existence of variations and types thereof could be detected at the same time for all the 64 types of test samples.

### Example 6

Preparation of a Substrate for Probe Matrices Partitioned by Patterns.

A glass substrate with an epoxy group introduced to the surface and with black matrices for probe matrices was prepared in accordance with the following procedure.

1. Introduction of an Epoxy Group to the Surface of the Substrate.

A glass substrate made of synthetic quartz (50 mm×50 mm) was first subjected to ultrasonic cleaning using a 2% sodium hydrate solution, and was then subjected to UV ozone processing to clean the surface. A 50% methanol solution containing 1% of silane coupling agent (trade name: KBM 403; manufactured by The Shin-Etsu Chemical Co., Ltd.) containing a silane compound having an epoxy group bonded thereto (γ-glycidoxypropyltrimethoxysilane) was stirred at room temperature for three hours to perform preliminary treatment for hydrolyzing the methoxy group in the silane compound. This solution already subjected to the hydrolysis treatment was applied to the surface of the above described clean substrate with a spin coater, and was heated and dried at 100° C. for 5 minutes to form a binding coating of the silane coupling agent on the surface of the substrate. Through the formation of this coating, the epoxy group contained in the silane compound was introduced to the surface of the substrate.

2. Formation of Black Matrices.

Then, A DEEP-UV resist containing carbon black (negative type resist for black matrices) (trade name: BK-739P; manufactured by Nippon Steel Chemical Co., Ltd.) was applied on the surface of the substrate with a spin coater so that the film thickness after curing was 5 μm, and it was heated for curing on a hotplate at 80° C. for 5 minutes. By proximity exposure using a DEEP-UV aligner, a pattern was exposed to light using as an exposure mask a mask for negatives with patterning applied to a region of 10 mm×10 mm so that the distance X between adjacent wells was 100 μm and the outer shape of the well was a square of 1 mm×1 mm. Then, development was carried out with a developer of inorganic aqueous alkaline solution using a spin drier, and the substrate was washed with pure water to remove the developer completely. Then, it was briefly dried using the spin drier, and was thereafter heated in a clean oven at 180° C. for 30 minutes to fully cure the resist. As a whole, a substrate with 400 wells arranged in a predetermined arrangement and black matrices (resist walls) partitioning adjacent wells was obtained. Furthermore, the internal volume of each well is calculated as 5 μl if the thickness of solution is 5 μm. Also, in the surface of the prepared black matrix, the angle of contact to water was 93 degrees and wettability with water was significantly low, while in the bottom of the well, the angle of contact to water was 35 degrees and the wettability with water was high.

3. Fixation of Probe DNA.

64 oligonucleotides of 18 mer with an amino group bound to the hydroxyl group of the five prime-end through a phosphate group and hexamethylene were prepared as DNA probes. The 64 probes are same as those prepared in Example 5 as to base sequences, but are different in the sense that an amino group is introduced in its five prime-end instead of a thiol group.

5 μl of solution of these DNA probes was injected into each well under a microscope, and was left in a humidified chamber to allow the probe bind to the substrate through the reaction between the amino group of the five primer-end and the epoxy group on the substrate.

### Example 7

Analysis of CDNA derived from the p 53 gene that has been prepared from mRNA, using the probe matrix substrate partitioned by the pattern that has been prepared in Example 6.

As in the case of Example 4, 64 types of labeled CDNAs were spotted in each probe region of 2 mm square as an arrangement of 8×8 spots as shown in FIG. 8, using a pin system array preparing apparatus.

A hybridization reaction was carried out by a method similar to that of example 4.

The obtained result was similar to that of Example 5.

What is claimed is:

1. A method of examining a reactivity of a first sample with a plurality of second samples having different properties from one another, comprising the steps of:
preparing a substrate with said first sample bound thereto in a defined region;
arranging said plurality of second samples within said region independently of one another; and
testing the reactivity of said first sample with each of said second samples.

2. The examination method according to claim 1, wherein said reactivity is a bonding capability between said first sample and said second samples.

3. The examination method according to claim 2, wherein said bonding capability is based on complementation of nucleic acid strands.

4. The examination method according to claim 1, wherein said first sample is originated from an organism, and said second samples have known properties.

5. The examination method according to claim 4, wherein said second samples are synthesized.

6. The examination method according to claim 5, wherein said first sample includes a nucleic acid originated from an organism and having an unknown base sequence, and said second samples include synthesized nucleic acids having known base sequences.

7. The examination method according to claim 6, wherein said first sample includes a set of mRNAs extracted from an organism.

8. The examination method according to claim 6, wherein said first sample includes a cDNA library synthesized based on mRNAs extracted from an organism.

9. The examination method according to claim 4, wherein said first sample includes a nucleic acid originated from an organism and having an unknown base sequence, and said second samples include synthesized chemicals.

10. The examination method according to claim 4, wherein said first sample includes a nucleic acid originated from an organism and having an unknown base sequence, and said second samples include purified proteins.

11. The examination method according to claim 1, wherein said first sample has a known property, and said second samples are originated from an organism.

12. The examination method according to claim 11, wherein said first sample includes a gene having a known sequence.

13. The examination method according to claim 11, wherein said first sample includes a cloned oncogene fragment, and said second samples include nucleic acids originated from an organism.

14. The examination method according to claim 1, wherein said first sample includes a protein fragment extracted from an organism, and said second samples include purified proteins of a single type.

15. The examination method according to claim 1, wherein said first sample includes a purified protein of a single type, and said second samples include protein fragments extracted from an organism.

16. The examination method according to claim 4, wherein said first sample includes a protein fragment originated from an organism, and said second samples include synthesized chemicals.

17. The examination method according to claim 4, wherein said first sample includes a purified protein of a single type, and said second samples include synthesized chemicals.

18. The examination method according to claim 1, wherein said first sample includes a synthesized chemical, and said second samples include nucleic acids extracted from an organism.

19. The examination method according to claim 1, wherein said first sample includes a synthesized chemical, and said second samples include protein fragments extracted from an organism.

20. The examination method according to claim 1, wherein said first sample is comprised of a plurality of samples having different properties, and each of said plurality of samples is bound to one of partitioned regions forming a matrix on the substrate.

21. The examination method according to claim 20, wherein said first sample includes nucleic acids originated from different biological species, tissues or cells.

22. The examination method according to claim 20, wherein said first sample includes proteins extracted from different biological species, tissues or cells.

23. The examination method according to claim 20, wherein the density of said matrix is 400/cm² or lower.

24. The examination method according to claim 20, wherein an array of spots of said second samples is arranged in each of said partitioned regions in a common arrangement.

25. The examination method according to claim 1, wherein said substrate is made of glass.

26. The examination method according to claim 1, wherein said first sample is fixed on the substrate by electrostatic bonds.

27. The examination method according to claim 1, wherein said first sample is fixed on the substrate by covalent bonds.

28. The examination method according to claim 27, wherein said first sample is bound to said substrate through a chemical reaction of maleimide groups introduced to a glass surface of the substrate with thiol groups possessed by said first sample.

29. The examination method according to claim 28, wherein said first sample includes a protein, and said thiol groups are cycteine groups of the protein.

30. The examination method according to claim 28, wherein said maleimide groups are introduced by introducing amino groups to the glass surface and then reacting said amino groups with N-(6-maleimidocaproyloxy)succinimide.

31. The examination method according to claim 28, wherein said maleimide groups are introduced by introducing amino groups to the glass surface and then reacting said amino groups with succinimidyl 4-(maleimidophenyl)butyrate.

32. The examination method according to claim 28, wherein said chemical reaction is a reaction between an epoxy group introduced to the glass surface of the substrate and an amino group possessed by said first sample.

33. The examination method according to claim 30, wherein said amino group is an amino group existing in a nucleic acid base.

34. The examination method according to claim 1, wherein said substrate has a surface previously partitioned by a wall member to define sections forming a matrix, and biological samples having different properties are previously bound to the respective sections as the first sample.

35. The examination method according to claim 34, wherein each of said sections has a hydrophobic wall portion and a hydrophilic bottom portion.

36. The examination method according to claim 35, wherein said wall member has a thickness in the range of 1 to 20 μm.

37. The examination method according to claim 1, wherein said second samples are arranged as spots with a diameter of 200 μm or smaller.

38. The examination method according to claim 1, wherein said second samples are arranged as spots with a density of 400/cm² or higher.

39. The examination method according to claim 1, wherein said second samples are supplied by an ink-jet method.

40. The examination method according to claim 39, wherein said second samples supplied by the ink-jet method include nucleic acids having a base pair length in the range of 2 to 5000 pairs.

41. The examination method according to claim 40, wherein said nucleic acids supplied by the ink-jet method are supplied as an aqueous solution having a concentration in the range of 0.05 to 500 μM.

42. The examination method according to claim 39, wherein said ink-jet method is a bubble jet method.

43. The examination method according to claim 1, wherein each of said second samples is supplied by contacting a pin with a solution of the sample and contacting said pin physically with said substrate.

44. The examination method according to claim 1, wherein each of said second samples is supplied by sucking a solution of the sample using a capillary and then contacting the tip of said capillary physically with the substrate.

45. A biological sample matrix, wherein two or more biological samples of different origins are bound to respective partitioned regions forming a matrix on a substrate.

46. The biological sample matrix according to claim 45, wherein said biological samples include cloned oncogene fragments.

47. The biological sample matrix according to claim 45, wherein said biological samples include mRNAs.

48. The biological sample matrix according to claim 45, wherein said biological samples include cDNAs.

49. The biological sample matrix according to claim 45, wherein said biological samples include a cDNA library.

50. The biological sample matrix according to claim 45, wherein said biological samples include two or more types of proteins having different conformations.

51. The biological sample matrix according to claim 45, wherein the density of said matrix is 400/cm² or lower.

52. The biological sample matrix according to claim 45, wherein said substrate is made of glass.

53. The biological sample matrix according to claim 45, wherein said biological samples are fixed on the substrate by electrostatic bonds.

54. The biological sample matrix according to claim 45, wherein said biological samples are fixed on the substrate by covalent bonds.

55. The biological sample matrix according to claim 54, wherein said biological samples are bound to said substrate through a chemical reaction of maleimide groups introduced to a glass surface of the substrate with thiol groups possessed by said biological samples.

56. The biological sample matrix according to claim 55, wherein said biological samples include proteins bound to the glass surface through a chemical reaction of maleimide groups introduced to the glass surface with thiol groups of cysteine residue of the protein.

57. The biological sample matrix according to claim 55, wherein said maleimide group is introduced by introducing amino groups to the glass surface and then reacting said amino groups with N-(6-maleimidocaproyloxy)succinimide.

58. The biological sample matrix according to claim 55, wherein said maleimide group is introduced by introducing amino groups to the glass surface and then reacting said amino groups with succinimidyl 4-(maleimidophenyl)butyrate.

59. The biological sample matrix according to claim 54, wherein said biological samples include nucleic acids bound to said substrate through a chemical reaction of epoxy groups introduced to a glass surface of the substrate with amino groups possessed by said nucleic acids.

60. The biological sample matrix according to claim 45, wherein said two or more biological samples are supplied on the respective partitioned regions on the substrate by an ink-jet method.

61. The biological sample matrix according to claim 45, wherein said substrate has a surface partitioned by a wall member to define sections forming a matrix, and said two or more biological samples of different origins are bound to the respective sections.

62. The biological sample matrix according to claim 61, wherein each of said sections has a hydrophobic wall portion and a hydrophilic bottom portion.

63. The biological sample matrix according to claim 62, wherein said wall member has a thickness in the range of 1 to 20 μm.

64. A method of detecting a complex formed between an oligonucleotide of which base sequence is known and a component having a capability of binding to said oligonucleotide, comprising the steps of:
preparing at least one oligonucleotide of which base sequence is known;
preparing at least two liquid test samples potentially containing a component having a capability of binding to said oligonucleotide;
binding said oligonucleotide as a probe to a predetermined region on a solid substrate to produce a detection substrate;
arranging a plurality of spots of said test samples at a predetermined amount to form an array of said test samples within said region with said oligonucleotide bound thereto;
detecting whether a complex between said oligonucleotide and said component is present or not for each of said plurality of spots; and
determining whether or not said component is contained in each of said liquid test samples, or how strong its binding capability to said oligonucleotide is, based on said detection.

65. The detection method according to claim 64, wherein said oligonucleotide bound to said detection substrate has a base sequence with a base length of 2 to 100.

66. The detection method according to claim 64, wherein said liquid test samples are solutions each containing at least one nucleic acid of which base sequence is unknown, detection is made whether a complex between said oligonucleotide and said nucleic acid is formed or not for each of said test samples to thereby determine whether or not said nucleic acid contains a base sequence complementary to the known base sequence of said oligonucleotide functioning as said component having a capability of binding to said oligonucleotide.

67. The detection method according to claim 66, wherein said nucleic acid contained in each of said liquid test samples includes a set of mRNAs extracted from an organic tissue.

68. The detection method according to claim 66, wherein said nucleic acid contained in each of said liquid test samples includes a CDNA library prepared based on a set of mRNAs extracted from an organic tissue.

69. The detection method according to claim 66, wherein said nucleic acid contained in each of said liquid test samples has a base length of 2 to 5000.

70. The detection method according to claim 64, wherein said liquid test samples are solutions each containing at lest one protein, the proteins contained in said test samples being different from one another.

71. The detection method according to claim 64, wherein said liquid test samples are solutions each containing at lest one chemical, the chemicals contained in said test samples being different from one another.

72. The detection method according to claim 64, wherein said liquid test samples are extracts from different biological species, tissues or cells.

73. The detection method according to claim 64, wherein a plurality of oligonucleotides having known base sequences different from one another are used as a probe, and said detection substrate has a plurality of predetermined sections arranged in a matrix form to which said oligonucleotides are to be bound, respectively.

74. The detection method according to claim 73, wherein said plurality of oligonucleotides are bound to said sections to constitute a matrix at a density of 400/cm2 or lower, said sections having the same area as one another.

75. The detection method according to claim 73, wherein said test samples are spotted in an array form in each of said sections to which said plurality of oligonucleotides having known base sequences different from one another are bound so that the spot positions in each section are arranged in the same way as one another.

76. The detection method according to claim 64, wherein said solid substrate used as said detection substrate is made of glass.

77. The detection method according to claim 64, wherein said oligonucleotide is fixed on the detection substrate by covalent bonds.

78. The detection method according to claim 77, wherein said oligonucleotide is fixed on the detection substrate by covalent bonds formed through a chemical reaction of maleimide groups introduced to a glass surface of the substrate used as said solid substrate with thiol (—SH) groups possessed by said oligonucleotide.

79. The detection method according to claim 78, wherein said maleimide groups introduced to the glass surface is formed by first introducing amino groups to the glass surface and then reacting N-(6-maleimidocaproyloxy)succinimide with the amino groups.

80. The detection method according to claim 78, wherein said maleimide groups introduced to the glass surface is formed by first introducing amino groups to the glass surface and then reacting succinimidyl 4-(maleimidophenyl)butyrate with the amino groups.

81. The detection method according to claim 77, wherein said oligonucleotide is fixed on the detection substrate by covalent bonds through a chemical reaction of epoxy groups introduced to a glass surface of the substrate used as said solid substrate with amino groups possessed by said oligonucleotide.

82. The detection method according to claim 64, wherein said detection substrate has a surface previously partitioned to form a plurality of sections, and two or more different types of oligonucleotides of which base sequences are known are previously bound to the sections, respectively, in a matrix form.

83. The detection method according to claim 82, wherein said sections previously formed on the surface of said detection substrate are separated from each other by a wall member and each section having a hydrophobic wall portion and a hydrophilic bottom portion section is hydrophilic.

84. The detection method according to claim 83, wherein said wall member has a thickness in the range of 1 to 20 μm.

85. The detection method according to claim 64, wherein each of the spots of said two or more test samples formed in each section has a diameter of 200 μm or lower.

86. The detection method according to claim 64, wherein the spots of said two or more test samples formed in each section is arranged at a density of 400/cm² or smaller.

87. The detection method according to claim 64, wherein each of the spots of said two or more test samples is formed by supplying a predetermined amount of a solution of said test samples by an ink-jet method.

88. The detection method according to claim 87, wherein said two or more test samples are spotted by an ink-jet method as a solution containing a nucleic acid with base length of 100 to 5000, respectively.

89. The detection method according to claim 88, wherein said two or more test samples are spotted by an ink-jet method as a solution containing a nucleic acid as a total concentrations of 0.05 to 500 μM, respectively.

90. The detection method according to claim 88, wherein said ink-jet method used for spotting is a bubble jet method.

91. The detection method according to claim 64, wherein the spots of test samples are formed by contacting a pin having a tip for collecting sample solutions with a solution of each test sample to allow the sample solution to adhere to the tip of said pin for taking a predetermined amount of the solution and then physically contacting the tip of said pin with a surface of the substrate to transfer said predetermined amount of the solution to the substrate surface.

92. The detection method according to claim 64, wherein the spots of test samples are formed by sucking a solution of each test sample using a capillary having a tip for sucking sample solutions thereinto and then physically contacting the tip of said capillary with a surface of the substrate to transfer a predetermined amount of the solution to the substrate surface.

93. A detection substrate with two or more oligonucleotides having known base sequences different from one another fixed on a solid substrate,
wherein said two or more oligonucleotides are bound and fixed on a plurality of predetermined sections, respectively, so that one oligonucleotide is present in each section, and said plurality of predetermined sections with oligonucleotides fixed therein are arranged in a matrix form on a surface of said solid substrate.

94. The detection substrate according to claim 93, wherein the known base sequence of each of said two or more oligonucleotides bound in predetermined sections has a base length of 2 to 60.

95. The detection substrate according to claim 93, wherein said plurality of predetermined sections are arranged in a matrix form on the surface of said solid substrate at a density of 400/cm² or lower.

96. The detection substrate according to claim 93, wherein said solid substrate is a glass substrate.

97. The detection substrate according to claim 93, wherein said oligonucleotides are fixed on the substrate surface by covalent bonds.

98. The detection substrate according to claim 97, wherein said oligonucleotides are fixed on the detection substrate by covalent bonds formed through a chemical reaction of maleimide groups introduced to a glass surface of the solid substrate with thiol groups possessed by said oligonucleotides.

99. The detection substrate according to claim 98, wherein said maleimide groups introduced to the glass surface are formed by first introducing amino groups to the glass surface and then reacting succinimidyl 4-(maleimidophenyl)butyrate with the amino groups.

100. The detection substrate according to claim 98, wherein said maleimide groups introduced to the glass surface are formed by first introducing amino groups to the glass surface and then reacting N-(6-maleimidocaproyloxy)succinimide with the amino groups.

101. The detection substrate according to claim 97, wherein said oligonucleotides are fixed on the detection substrate by covalent bonds through a chemical reaction of epoxy groups introduced to a glass surface of the solid substrate with amino groups possessed by said oligonucleotides.

102. The detection substrate according to claim 93, wherein said two or more oligonucleotides are fixed in each of said predetermined sections such that one oligonucleotide is present in each section by supplying said two or more oligonucleotides in each of said predetermined sections in a matrix form by printing them by an ink-jet process.

103. The detection substrate according to claim 93, wherein said two or more oligonucleotides are bound to said plurality of predetermined sections previously partitioned in a matrix form.

104. The detection substrate according to claim 103, wherein said sections previously formed in a matrix form on the substrate surface are separated from each other by a wall member and each section has a hydrophobic wall portion and a hydrophilic bottom portion.

105. The detection substrate according to claim 104, wherein said wall member has a thickness in the range of 1 to 20 μm.

106. The detection substrate according to claim 104, wherein two or more oligonucleotides are fixed to said previously formed sections in a matrix form by an ink-jet method so that said two or more oligonucleotides are supplied only on the bottom portion of each section.

107. A method of preparing a detection substrate with two or more oligonucleotides having known base sequences different from one another fixed on a solid substrate, comprising:
preparing a solid substrate having a surface previously partitioned into a plurality of sections in a matrix form,
supplying a predetermined amount of two or more oligonucleotides in said predetermined sections so that only one type of said oligonucleotides is present in each section by an ink-jet method, and
fixing the supplied oligonucleotides in the predetermined sections.


SEQUENCE LISTING (64 sequences)

SEQ ID NO: 1 - DNA, 18 bp, Artificial sequence
  Sample oligonucleotide
gatgggactcaagttcat

SEQ ID NO: 2 - DNA, 18 bp, Artificial sequence
  Sample oligonucleotide
gatgggactcaggttcat

SEQ ID NO: 3 - DNA, 18 bp, Artificial sequence
  Sample oligonucleotide
gatgggactcacgttcat

SEQ ID NO: 4 - DNA, 18 bp, Artificial sequence
  Sample oligonucleotide
gatgggactcatgttcat

SEQ ID NO: 5 - DNA, 18 bp, Artificial sequence
  Sample oligonucleotide
gatgggactcgagttcat

SEQ ID NO: 6 - DNA, 18 bp, Artificial sequence
  Sample oligonucleotide
gatgggactcgggttcat

SEQ ID NO: 7 - DNA, 18 bp, Artificial sequence
  Sample oligonucleotide
gatgggactcgcgttcat

SEQ ID NO: 8 - DNA, 18 bp, Artificial sequence
  Sample oligonucleotide
gatgggactcgtgttcat

SEQ ID NO: 9 - DNA, 18 bp, Artificial sequence
  Sample oligonucleotide
gatgggactccagttcat

SEQ ID NO: 10 - DNA, 18 bp, Artificial sequence
  Sample oligonucleotide
gatgggactccggttcat

SEQ ID NO: 11 - DNA, 18 bp, Artificial sequence
  Sample oligonucleotide
gatgggactcccgttcat

SEQ ID NO: 12 - DNA, 18 bp, Artificial sequence
  Sample oligonucleotide
gatgggactcctgttcat

SEQ ID NO: 13 - DNA, 18 bp, Artificial sequence
  Sample oligonucleotide
gatgggactctagttcat

SEQ ID NO: 14 - DNA, 18 bp, Artificial sequence
  Sample oligonucleotide
gatgggactctggttcat

SEQ ID NO: 15 - DNA, 18 bp, Artificial sequence
  Sample oligonucleotide
gatgggactctcgttcat

SEQ ID NO: 16 - DNA, 18 bp, Artificial sequence
  Sample oligonucleotide
gatgggactcttgttcat

SEQ ID NO: 17 - DNA, 18 bp, Artificial sequence
  Sample oligonucleotide
gatggggctcaagttcat

SEQ ID NO: 18 - DNA, 18 bp, Artificial sequence
  Sample oligonucleotide
gatggggctcaggttcat

SEQ ID NO: 19 - DNA, 18 bp, Artificial sequence
  Sample oligonucleotide
gatggggctcacgttcat

SEQ ID NO: 20 - DNA, 18 bp, Artificial sequence
  Sample oligonucleotide
gatggggctcatgttcat

SEQ ID NO: 21 - DNA, 18 bp, Artificial sequence
  Sample oligonucleotide
gatggggctcgagttcat

SEQ ID NO: 22 - DNA, 18 bp, Artificial sequence
  Sample oligonucleotide
gatggggctcgggttcat

SEQ ID NO: 23 - DNA, 18 bp, Artificial sequence
  Sample oligonucleotide
gatggggctcgcgttcat

SEQ ID NO: 24 - DNA, 18 bp, Artificial sequence
  Sample oligonucleotide
gatggggctcgtgttcat

SEQ ID NO: 25 - DNA, 18 bp, Artificial sequence
  Sample oligonucleotide
gatggggctccagttcat

SEQ ID NO: 26 - DNA, 18 bp, Artificial sequence
  Sample oligonucleotide
gatggggctccggttcat

SEQ ID NO: 27 - DNA, 18 bp, Artificial sequence
  Sample oligonucleotide
gatggggctcccgttcat

SEQ ID NO: 28 - DNA, 18 bp, Artificial sequence
  Sample oligonucleotide
gatggggctcctgttcat

SEQ ID NO: 29 - DNA, 18 bp, Artificial sequence
  Sample oligonucleotide
gatggggctctagttcat

SEQ ID NO: 30 - DNA, 18 bp, Artificial sequence
  Sample oligonucleotide
gatggggctctggttcat

SEQ ID NO: 31 - DNA, 18 bp, Artificial sequence
  Sample oligonucleotide
gatggggctctcgttcat

SEQ ID NO: 32 - DNA, 18 bp, Artificial sequence
  Sample oligonucleotide
gatggggctcttgttcat

SEQ ID NO: 33 - DNA, 18 bp, Artificial sequence
  Sample oligonucleotide
gatgggcctcaagttcat

SEQ ID NO: 34 - DNA, 18 bp, Artificial sequence
  Sample oligonucleotide
gatgggcctcaggttcat

SEQ ID NO: 35 - DNA, 18 bp, Artificial sequence
  Sample oligonucleotide
gatgggcctcacgttcat

SEQ ID NO: 36 - DNA, 18 bp, Artificial sequence
  Sample oligonucleotide
gatgggcctcatgttcat

SEQ ID NO: 37 - DNA, 18 bp, Artificial sequence
  Sample oligonucleotide
gatgggcctcgagttcat

SEQ ID NO: 38 - DNA, 18 bp, Artificial sequence
  Sample oligonucleotide
gatgggcctcgggttcat

SEQ ID NO: 39 - DNA, 18 bp, Artificial sequence
  Sample oligonucleotide
gatgggcctcgcgttcat

SEQ ID NO: 40 - DNA, 18 bp, Artificial sequence
  Sample oligonucleotide
gatgggcctcgtgttcat

SEQ ID NO: 41 - DNA, 18 bp, Artificial sequence
  Sample oligonucleotide
gatgggcctccagttcat

SEQ ID NO: 42 - DNA, 18 bp, Artificial sequence
  Sample oligonucleotide
gatgggcctccggttcat

SEQ ID NO: 43 - DNA, 18 bp, Artificial sequence
  Sample oligonucleotide
gatgggcctcccgttcat

SEQ ID NO: 44 - DNA, 18 bp, Artificial sequence
  Sample oligonucleotide
gatgggcctcctgttcat

SEQ ID NO: 45 - DNA, 18 bp, Artificial sequence
  Sample oligonucleotide
gatgggcctctagttcat

SEQ ID NO: 46 - DNA, 18 bp, Artificial sequence
  Sample oligonucleotide
gatgggcctctggttcat

SEQ ID NO: 47 - DNA, 18 bp, Artificial sequence
  Sample oligonucleotide
gatgggcctctcgttcat

SEQ ID NO: 48 - DNA, 18 bp, Artificial sequence
  Sample oligonucleotide
gatgggcctcttgttcat

SEQ ID NO: 49 - DNA, 18 bp, Artificial sequence
  Sample oligonucleotide
gatgggtctcaagttcat

SEQ ID NO: 50 - DNA, 18 bp, Artificial sequence
  Sample oligonucleotide
gatgggtctcaggttcat

SEQ ID NO: 51 - DNA, 18 bp, Artificial sequence
  Sample oligonucleotide
gatgggtctcacgttcat

SEQ ID NO: 52 - DNA, 18 bp, Artificial sequence
  Sample oligonucleotide
gatgggtctcatgttcat

SEQ ID NO: 53 - DNA, 18 bp, Artificial sequence
  Sample oligonucleotide
gatgggtctcgagttcat

SEQ ID NO: 54 - DNA, 18 bp, Artificial sequence
  Sample oligonucleotide
gatgggtctcgggttcat

SEQ ID NO: 55 - DNA, 18 bp, Artificial sequence
  Sample oligonucleotide
gatgggtctcgcgttcat

SEQ ID NO: 56 - DNA, 18 bp, Artificial sequence
  Sample oligonucleotide
gatgggtctcgtgttcat

SEQ ID NO: 57 - DNA, 18 bp, Artificial sequence
  Sample oligonucleotide
gatgggtctccagttcat

SEQ ID NO: 58 - DNA, 18 bp, Artificial sequence
  Sample oligonucleotide
gatgggtctccggttcat

SEQ ID NO: 59 - DNA, 18 bp, Artificial sequence
  Sample oligonucleotide
gatgggtctcccgttcat

SEQ ID NO: 60 - DNA, 18 bp, Artificial sequence
  Sample oligonucleotide
gatgggtctcctgttcat

SEQ ID NO: 61 - DNA, 18 bp, Artificial sequence
  Sample oligonucleotide
gatgggtctctagttcat

SEQ ID NO: 62 - DNA, 18 bp, Artificial sequence
  Sample oligonucleotide
gatgggtctctggttcat

SEQ ID NO: 63 - DNA, 18 bp, Artificial sequence
  Sample oligonucleotide
gatgggtctctcgttcat

SEQ ID NO: 64 - DNA, 18 bp, Artificial sequence
  Sample oligonucleotide
gatgggtctcttgttcat